(19) United States
(12) Patent Application Publication
(10) Pub. No.: US 2026/0253696 A1
(43) Pub. Date: Aug. 27, 2026

(54) SYSTEM

(71) Applicant: SoftBank Group Corp., Tokyo (JP)

(72) Inventor: Akiyoshi KUDO, Tokyo (JP)

(73) Assignee: SoftBank Group Corp., Tokyo (JP)

(21) Appl. No.: 19/531,714

(22) Filed: Feb. 6, 2026

(30) Foreign Application Priority Data
Feb. 21, 2025 (JP) 2025-027010

(51) Int. Cl. G16H 15/00; A61B 5/00; A61B 5/11; A61B 5/16; G06F 40/284; G06F 40/30; G06F 40/40; G10L 17/02; G10L 17/04; G10L 17/18; G10L 25/63

(52) U.S. Cl. CPC G16H 15/00; A61B 5/0022; A61B 5/1112; A61B 5/165; A61B 5/4803; G06F 40/284; G06F 40/30; G06F 40/40; G10L 17/02; G10L 17/04; G10L 17/18; G10L 25/63

(57) ABSTRACT

The system according to the embodiment comprises a recording unit, a conversion unit, an analysis unit, and a provision unit. The recording unit records a user's voice. The conversion unit converts the voice recorded by the recording unit into a character string. The analysis unit analyzes the character string converted by the conversion unit. The provision unit provides the analysis result obtained by the analysis unit as a report.

### CROSS-REFERENCE TO RELATED APPLICATION

The present application claims priority to and incorporates by reference the entire contents of Japanese Patent Application No. 2025-027010 filed in Japan on Feb. 21, 2025.

### BACKGROUND OF THE INVENTION

### 1. Field of the Invention

The technology of this disclosure relates to a system.

### 2. Description of the Related Art

Japanese Patent Application Laid-open No. 2022-180282 discloses a persona chatbot control method executed by at least one processor, comprising: receiving a user utterance, adding the user utterance to a prompt containing instructions related to the character of the chatbot, encoding the prompt, inputting the encoded prompt into a language model, and generating a chatbot utterance in response to the user utterance.

In conventional technology, there has been a problem that it is difficult to obtain specific feedback for improving the quality of dialogue.

### SUMMARY OF THE INVENTION

The system according to the embodiment comprises a recording unit, a conversion unit, an analysis unit, and a provision unit. The recording unit records a user's voice. The conversion unit converts the voice recorded by the recording unit into a character string. The analysis unit analyzes the character string converted by the conversion unit. The provision unit provides the analysis result obtained by the analysis unit as a report.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a conceptual diagram showing an example configuration of a data processing system according to the first embodiment;

FIG. 2 is a conceptual diagram showing an example of main functions of a data processing device and a smart device according to the first embodiment;

FIG. 3 is a conceptual diagram showing an example configuration of a data processing system according to the second embodiment;

FIG. 4 is a conceptual diagram showing an example of main functions of a data processing device and smart glasses according to the second embodiment;

FIG. 5 is a conceptual diagram showing an example configuration of a data processing system according to the third embodiment;

FIG. 6 is a conceptual diagram showing an example of main functions of a data processing device and a headset-type terminal according to the third embodiment;

FIG. 7 is a conceptual diagram showing an example configuration of a data processing system according to the fourth embodiment;

FIG. 8 is a conceptual diagram showing an example of main functions of a data processing device and a robot according to the fourth embodiment;

FIG. 9 shows an emotion map where multiple emotions are mapped; and

FIG. 10 shows an emotion map where multiple emotions are mapped.

### DETAILED DESCRIPTION OF THE PREFERRED EMBODIMENTS

Hereinafter, an example of an embodiment of the system related to the technology disclosed herein will be described with reference to the attached drawings.

First, the terminology used in the following description will be explained.

In the following embodiments, a processor denoted by a reference numeral (hereinafter simply referred to as “processor”) may be a single computing device or a combination of multiple computing devices. The processor may be a single type of computing device or a combination of multiple types of computing devices. Examples of computing devices include a CPU (Central Processing Unit), GPU (Graphics Processing Unit), GPGPU (General-Purpose computing on Graphics Processing Units), APU (Accelerated Processing Unit), or TPU (Tensor Processing Unit), among others.

In the following embodiments, a RAM (Random Access Memory) denoted by a reference numeral is a memory where information is temporarily stored and used as a work memory by the processor.

In the following embodiments, a storage denoted by a reference numeral is one or more non-volatile storage devices for storing various programs and parameters. Examples of non-volatile storage devices include flash memory (SSD (Solid State Drive)), magnetic disks (e.g., hard disks), or magnetic tapes, among others.

In the following embodiments, a communication I/F (Interface) denoted by a reference numeral is an interface including a communication processor and an antenna, among others. The communication I/F manages communication between multiple computers. Examples of communication standards applicable to the communication I/F include wireless communication standards such as 5G (5th Generation Mobile Communication System), Wi-Fi (registered trademark), or Bluetooth (registered trademark), among others.

In the following embodiments, “A and/or B” means “at least one of A and B.” In other words, “A and/or B” means it may be only A, only B, or a combination of A and B. Moreover, when expressing three or more items connected by “and/or,” the same concept as “A and/or B” applies.

### First Embodiment

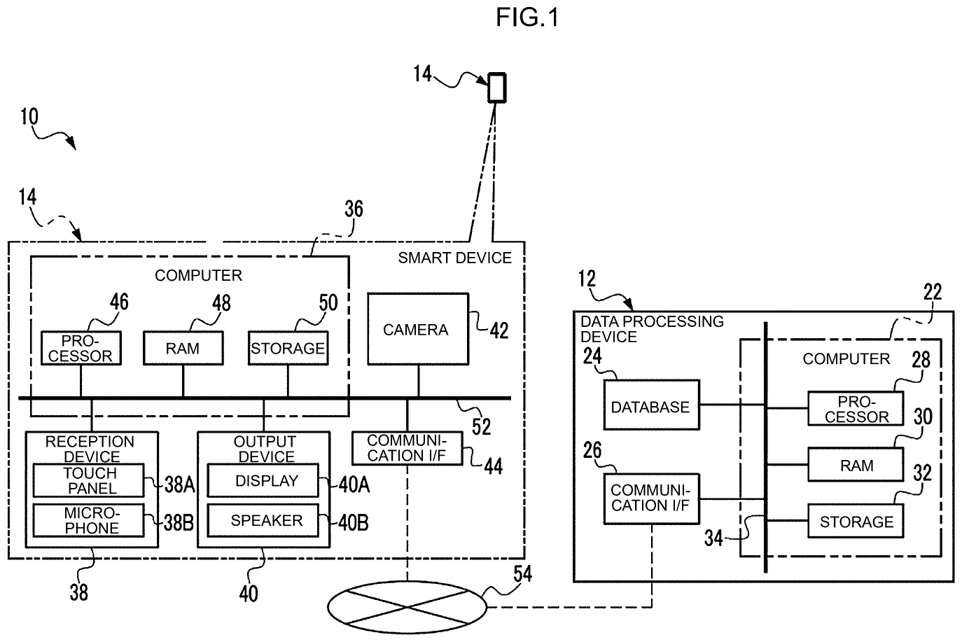

FIG. 1 shows an example configuration of a data processing system 10 according to the first embodiment.

As shown in FIG. 1, the data processing system 10 comprises a data processing device 12 and a smart device 14. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN (Wide Area Network) and/or a LAN (Local Area Network), among others.

The smart device 14 comprises a computer 36, a reception device 38, an output device 40, a camera 42, and a communication I/F 44. The computer 36 comprises a processor 46, RAM 48, and storage 50. The processor 46, RAM 48, and storage 50 are connected to a bus 52. The reception device 38, output device 40, and camera 42 are also connected to the bus 52.

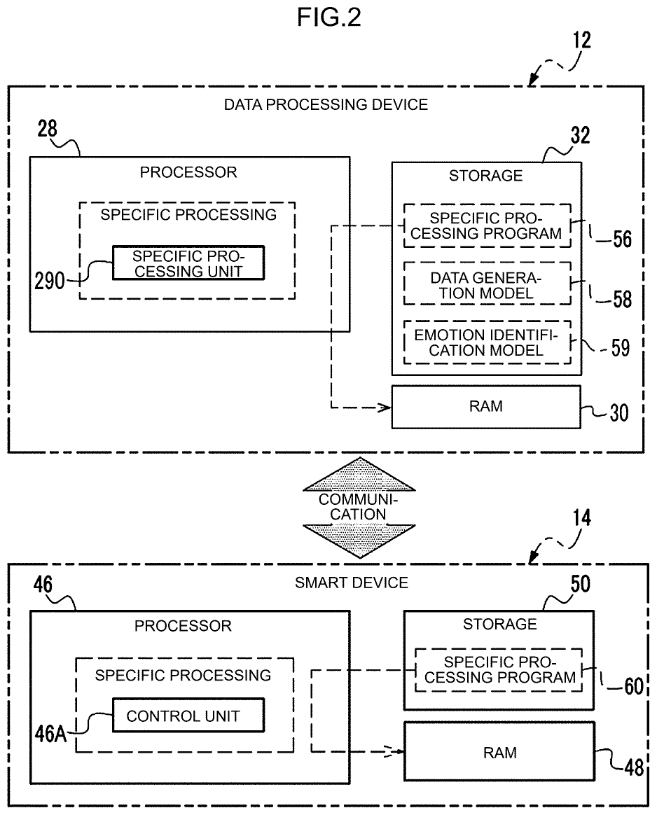

The reception device 38 comprises a touch panel 38A and a microphone 38B, among others, and accepts user input. The touch panel 38A accepts user input by detecting contact from an indicating object (e.g., a pen or finger). The microphone 38B accepts user input by detecting the user's voice. The control unit 46A sends data indicating user input accepted by the touch panel 38A and microphone 38B to the data processing device 12. The data processing device 12 has a specific processing unit 290 (see FIG. 2) that acquires data indicating user input.

The output device 40 comprises a display 40A and a speaker 40B, among others, and presents data to the user by outputting it in a perceptible form (e.g., audio and/or text). The display 40A displays visible information such as text and images according to instructions from the processor 46. The speaker 40B outputs audio according to instructions from the processor 46. The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors.

The communication I/F 44 is connected to the network 54. The communication I/F 44 and 26 manage the exchange of various information between the processor 46 and the processor 28 via the network 54.

FIG. 2 shows an example of the main functions of the data processing device 12 and the smart device 14.

As shown in FIG. 2, specific processing is performed in the data processing device 12 by the processor 28. The storage 32 stores a specific processing program 56. The specific processing program 56 is an example of a “program” related to the technology disclosed herein. The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart device 14, specific processing is performed by the processor 46. The storage 50 stores a specific processing program 60. The specific processing program 60 is used in conjunction with the specific processing program 56 by the data processing system 10. The processor 46 reads the specific processing program 60 from the storage 50 and executes it on the RAM 48. The specific processing is realized by the processor 46 operating as a control unit 46A according to the specific processing program 60 executed on the RAM 48. The smart device 14 may also have similar data generation models and emotion identification models as the data generation model 58 and emotion identification model 59, and perform the same processing as the specific processing unit 290 using these models.

Other devices besides the data processing device 12 may have the data generation model 58. For example, a server device (e.g., a generation server) may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.). Next, an example of processing by the data processing system 10 according to the first embodiment will be described.

### Example of the Embodiment

The conversation quality improvement system according to the embodiment of the present invention is a system for improving the quality of 1-on-1 conversations and conversations with acquaintances. This system records the user's voice, converts it into a character string for recording, analyzes statements that impair psychological safety, and generates a report. The user can read the report to learn about issues in their conversation and ways to improve. In the paid version, more than ten records can be saved, and the growth trend of communication skills related to psychological safety can be confirmed. For example, the user records their own voice (voiceprint). At this time, the user's voice is recorded in detail and used for subsequent processing. Next, the system stores conversations such as 1-on-1 meetings, converts the voice into a character string, and records it. For example, by saving the content of the conversation as text, it can be reviewed later. The system analyzes the recorded character string and identifies statements that impair psychological safety. For example, it detects aggressive words or negative expressions. This allows the user to understand the issues in their own statements. Next, the system provides the analysis result as a report. The user can read the report to learn about issues in their conversation and ways to improve. For example, the user can specifically learn what kind of language impairs psychological safety and how to improve it. In the paid version, more than ten records can be saved. In addition, the growth trend of communication skills related to psychological safety can be confirmed. For example, by comparing with past reports, the user can feel their own growth. As a result, the conversation quality improvement system can improve the quality of the user's conversations. Specifically, the conversation quality improvement system obtains the user's voice data as high-quality digital audio files such as PCM or WAV format using the recording unit, and the recording unit dynamically adjusts the sampling rate and bit depth to optimize noise resistance and sound quality. The recording unit extracts features such as MFCC (Mel-frequency cepstral coefficients) and spectrograms from the audio data and passes them to the conversion unit as pre-processing data for speech recognition. The conversion unit uses convolutional neural networks (CNN), recurrent neural networks (RNN), or Transformer-based speech recognition models, takes audio tensors (e.g., 1-second audio waveform of length 16000×1 or spectrogram of 128 dimensions×100 frames) as input, and generates a character string sequence (e.g., UTF-8 encoded string such as “Thank you for today”) as output. The conversion unit combines acoustic and language models and uses beam search or CTC (Connectionist Temporal Classification) decoders to determine the optimal character string sequence. The analysis unit takes the character string data obtained from the conversion unit as input and uses large-scale language models for natural language processing (e.g., Transformer-based encoder-decoder models) or rule-based text analysis engines to detect expressions in each statement that impair psychological safety (e.g., “You are useless”, “Can't you even do that?”). The analysis unit receives tokenized character string arrays (e.g., subword ID sequences or word vector sequences) as input and generates labels for each statement (e.g., 0=safe, 1=aggressive, 2=negative) or scores (e.g., psychological safety risk 0.85) as output. These outputs are used for subsequent processing such as threshold judgment, heatmap display, and feedback generation for each statement. The provision unit automatically generates structured reports such as PDF, HTML, or JSON based on the output of the analysis unit and visualizes them on the user interface. The report includes risk assessment for each statement, examples of improvement, and comparison graphs with the past (e.g., time-series transition of psychological safety scores). In the paid version, more than ten conversation records and analysis results are saved in the database, and a dashboard function is provided to visualize the growth trend for each user. For example, it can display a graph of psychological safety score trends for user A over the past six months, a heatmap of statement tendencies, and a history of improvement advice. As a technical effect, this system enables high-speed processing of large-scale data, quantitative evaluation of statement tendencies, and generation of individually optimized feedback, which are difficult to achieve with manual recording, analysis, and feedback by humans. It goes beyond conventional automation to realize essential improvements in computer technology, such as high-precision conversation quality evaluation and improvement support by AI. Application fields include support for 1-on-1 interviews in companies, communication guidance in educational settings, psychological care in medical and welfare fields, and quality management in customer support, among others.

The conversation quality improvement system according to the embodiment comprises a recording unit, a conversion unit, an analysis unit, and a provision unit. The recording unit records the user's voice. The user's voice may include, for example, conversation voice, instruction voice, emotional expression voice, but is not limited to such examples. The recording unit, for example, records the user's voice in high quality and uses it for subsequent processing. The conversion unit converts the voice recorded by the recording unit into a character string. Specific methods for converting into a character string include, for example, speech recognition technology, conversion accuracy, conversion algorithms, but are not limited to such examples. For example, the conversion unit uses speech recognition technology to convert voice into a character string. In addition, the conversion unit may apply specific algorithms to improve conversion accuracy. The analysis unit analyzes the character string converted by the conversion unit. Specific methods for analysis include, for example, text analysis technology, analysis items, analysis algorithms, but are not limited to such examples. For example, the analysis unit uses text analysis technology to analyze the character string and identify statements that impair psychological safety. The provision unit provides the analysis result obtained by the analysis unit as a report. Specific formats and provision methods for the report include, for example, PDF format, web page format, email transmission, but are not limited to such examples. For example, the provision unit provides the analysis result as a report in PDF format. The provision unit may also provide the report in web page format. As a result, the conversation quality improvement system according to the embodiment can improve the quality of the user's conversations. Specifically, the conversation quality improvement system records the user's voice at a high sampling rate such as 16 kHz/24 bit using the recording unit, applies preprocessing such as noise removal and normalization to the audio signal, and then passes the data to the conversion unit. The conversion unit takes an audio tensor (e.g., waveform data of 16000 samples for 1 second) as input, uses convolutional neural networks (CNN), autoregressive models (RNN, LSTM), or Transformer-based speech recognition models to extract acoustic features (e.g., MFCC, spectrogram), and performs decoding at the phoneme, word, and sentence levels. The conversion unit uses beam search or CTC decoders to select the optimal character string sequence from multiple candidates and generates a UTF-8 encoded character string (e.g., “Thank you for today”) as output. The analysis unit tokenizes the character string obtained from the conversion unit and uses BERT, Transformer-based large-scale language models, or rule-based text analysis engines to detect expressions in each statement that impair psychological safety (e.g., “You are useless”, “Can't you even do that?”). The analysis unit receives token ID sequences or word vector sequences as input and generates labels for each statement (e.g., 0=safe, 1=aggressive, 2=negative) or scores (e.g., psychological safety risk 0.85) as output. These outputs are used for subsequent processing such as threshold judgment, heatmap display, and feedback generation for each statement. The provision unit automatically generates structured reports such as PDF, HTML, or JSON based on the output of the analysis unit and visualizes them on the user interface. The report includes risk assessment for each statement, examples of improvement, and comparison graphs with the past (e.g., time-series transition of psychological safety scores). As a technical effect, this system enables high-speed processing of large-scale data, quantitative evaluation of statement tendencies, and generation of individually optimized feedback, which are difficult to achieve with manual recording, analysis, and feedback by humans. It goes beyond conventional automation to realize essential improvements in computer technology, such as high-precision conversation quality evaluation and improvement support by AI. Application fields include support for 1-on-1 interviews in companies, communication guidance in educational settings, psychological care in medical and welfare fields, and quality management in customer support, among others.

The recording unit can record the user's voiceprint. The voiceprint may include, for example, voiceprint features, recording format, recording accuracy, but is not limited to such examples. The recording unit, for example, records the user's voiceprint with high accuracy. The recording unit may also record the features of the voiceprint in detail. By recording the user's voiceprint, it is possible to perform voice recording tailored to individual users. Some or all of the above-described processing in the recording unit may be performed using AI or without using AI. For example, the recording unit can input the user's voiceprint to AI and have the AI extract the features of the voiceprint. Specifically, the recording unit acquires the user's audio waveform data (e.g., 16 kHz, 16 bit, 5 seconds of PCM data), extracts multidimensional feature vectors for each frame (e.g., 13-dimensional MFCC×500 frames) such as MFCC (Mel-frequency cepstral coefficients), spectral envelope, fundamental frequency (F0), formant, and zero-crossing rate. The recording unit vectorizes these features and stores them in the database as a unique voiceprint template for each user. When using AI, the recording unit inputs audio tensors (e.g., spectrogram of 128 dimensions×100 frames) to convolutional neural networks (CNN) or self-supervised learning models (e.g., SimCLR, BYOL) and generates a 128-dimensional embedding vector (voiceprint feature) as output. For example, by inputting audio waveforms such as “Hello, this is Tanaka” or “Thank you for your hard work” to AI, output examples such as a 128-dimensional vector [0.12, −0.34, . . . , 0.56] or [0.08, 0.22, . . . , −0.41] can be obtained. These voiceprint features are used as identifiers for user authentication or individually optimized voice processing. As subsequent processing, the recording unit uses the extracted voiceprint features to dynamically optimize voice recording parameters (e.g., noise suppression level, emotion estimation model selection) for each user. As a technical effect, by using AI to extract and manage voiceprints in a high-dimensional feature space, the recording unit enables highly individualized voice processing and secure authentication compared to conventional simple voice recording methods, greatly improving the accuracy and reliability of the entire conversation quality improvement system. Application fields include conversation recording requiring personal authentication, record management for each patient in medical and welfare settings, and individualized instruction support in education.

The conversion unit can convert the recorded voice into a character string. Specific methods for converting into a character string include, for example, speech recognition technology, conversion accuracy, conversion algorithms, but are not limited to such examples. The conversion unit, for example, uses speech recognition technology to convert voice into a character string. The conversion unit may also apply specific algorithms to improve conversion accuracy. By converting the recorded voice into a character string, subsequent confirmation and analysis become easier. Some or all of the above-described processing in the conversion unit may be performed using AI or without using AI. For example, the conversion unit can input the recorded voice to AI and have the AI perform the conversion into a character string. Specifically, the conversion unit takes audio tensors received from the recording unit (e.g., waveform data of 16000 samples for 1 second or spectrogram of 128 dimensions×100 frames) as input, uses convolutional neural networks (CNN), recurrent neural networks (RNN), or Transformer-based speech recognition models to extract acoustic features. The conversion unit integrates acoustic and language models and uses beam search or CTC (Connectionist Temporal Classification) decoders to generate the optimal character string sequence from the audio signal. For example, by inputting audio waveforms such as “Good morning” or “Let's start the meeting” to AI, output examples such as UTF-8 encoded character strings “Good morning” or “Let's start the meeting” can be obtained. The conversion unit may apply algorithms such as noise suppression, speaker adaptation, and utilization of contextual information to improve conversion accuracy. As subsequent processing, the character string output by the conversion unit is passed to the analysis unit and used for psychological safety evaluation and analysis of statement tendencies. As a technical effect, by using AI to achieve high-precision speech recognition, the conversion unit greatly improves robustness against noisy environments and diverse speakers and speech patterns compared to conventional rule-based or simple dictionary matching methods, enhancing the reliability and practicality of the entire conversation quality improvement system. Application fields include creation of minutes for business meetings, lesson recording in educational settings, and medical record keeping in healthcare.

The analysis unit can identify statements that impair psychological safety. Statements that impair psychological safety may include, for example, aggressive words, negative expressions, but are not limited to such examples. The analysis unit, for example, uses text analysis technology to analyze character strings and identify statements that impair psychological safety. The analysis unit may also apply specific algorithms to identify statements that impair psychological safety with high accuracy. By identifying statements that impair psychological safety, the user can understand the issues in their own statements. Some or all of the above-described processing in the analysis unit may be performed using AI or without using AI. For example, the analysis unit can input character strings to AI and have the AI identify statements that impair psychological safety. Specifically, the analysis unit takes character string data received from the conversion unit (e.g., statement texts such as “You are useless”, “Can't you even do that?”) as input, uses BERT, Transformer-based large-scale language models, or rule-based text analysis engines to detect expressions in each statement that impair psychological safety. The analysis unit receives tokenized character string arrays (e.g., subword ID sequences or word vector sequences) as input and generates labels for each statement (e.g., 0=safe, 1=aggressive, 2=negative) or scores (e.g., psychological safety risk 0.85) as output. For example, by inputting the character string “You are useless” to AI, output examples such as “Aggressive (score 0.92)” or “Psychological safety risk 0.85” can be obtained. As subsequent processing, the analysis unit uses the output labels and scores to generate feedback for each statement, create reports, and display heatmaps. As a technical effect, by using AI to achieve high-precision psychological safety evaluation, the analysis unit enables flexible and objective statement evaluation that accommodates contextual dependence and diverse expression patterns, greatly improving the reliability and usefulness of the entire conversation quality improvement system compared to conventional simple keyword detection or manual evaluation. Application fields include support for 1-on-1 interviews in companies, communication guidance in educational settings, and psychological care in medical and welfare fields.

The provision unit can provide the analysis result as a report. Specific formats and provision methods for the report include, for example, PDF format, web page format, email transmission, but are not limited to such examples. The provision unit, for example, provides the analysis result as a report in PDF format. The provision unit may also provide the report in web page format. By providing the analysis result as a report, the user can learn about issues in their conversation and ways to improve. Some or all of the above-described processing in the provision unit may be performed using AI or without using AI. For example, the provision unit can input the analysis result to AI and have the AI generate the report. Specifically, the provision unit takes structured data such as labels and scores for each statement, statement text, and time-series data received from the analysis unit as input, and uses a template engine or large-scale language model (e.g., Transformer-based text generation model) to automatically generate reports in PDF, HTML, or JSON format. For example, by inputting data such as “Statement 1: Aggressive (score 0.92)”, “Statement 2: Safe (score 0.12)” to AI, output examples such as sentences like “Statement 1 contains expressions that impair psychological safety. Improvement example: Rephrase as ‘○○’.” or risk assessment graphs for each statement, and comparison charts with the past can be obtained. As subsequent processing, the provision unit implements functions such as making the generated report downloadable as a PDF file, displaying it interactively on a web page, or automatically sending it by email. As a technical effect, by using AI to automatically generate and distribute structured reports from analysis results, the provision unit achieves significant efficiency, accuracy, and individual optimization compared to manual report creation, enabling users to quickly and accurately learn about issues in their conversation and ways to improve. Application fields include support for 1-on-1 interviews in companies, communication guidance in educational settings, psychological care in medical and welfare fields, and quality management in customer support.

The provision unit can provide information for the user to learn by reading the report. Information for learning may include, for example, identification of improvement points, specific advice, but is not limited to such examples. The provision unit, for example, includes identification of improvement points in the report. The provision unit may also include specific advice in the report. By having the user read the report and learn, the quality of conversation can be improved. Some or all of the above-described processing in the provision unit may be performed using AI or without using AI. For example, the provision unit can input the content of the report to AI and have the AI generate information for learning. Specifically, the provision unit provides structured data such as labels for each statement (e.g., 0=safe, 1=aggressive, 2=negative), scores (e.g., psychological safety risk 0.85), statement text, and time-series data received from the analysis unit as input data to AI. Examples of input data include “Statement 1: Aggressive (score 0.92)”, “Statement 2: Safe (score 0.12)”, or “Statement 3: Negative (score 0.75)”. Based on these data, AI generates output such as identification of improvement points for each statement (e.g., “Statement 1 contains aggressive expressions. Rephrase as ‘○○’.”), specific advice (e.g., “Increase expressions that respect the other person's opinion.”), risk assessment graphs, and comparison charts with the past. Output data formats include natural language sentences, graph image data, and structured advice data in JSON format. For example, AI generates sentences such as “Statement 1 contains expressions that impair psychological safety. Improvement example: Rephrase as ‘○○’.” or “The proportion of aggressive statements in the past six 1-on-1 interviews is decreasing.” As subsequent processing, the provision unit automatically converts the learning information generated by AI into report formats such as PDF, HTML, or dashboard UI and presents it to the user. Furthermore, the provision unit can collect the user's browsing history and feedback and reflect it in the report generation algorithm for future reports. As a technical effect, by using AI to automatically generate and provide individually optimized learning information from statement analysis results, the provision unit enables high-precision feedback and continuous learning support tailored to each user's issues, greatly improving the usefulness and user experience of the entire conversation quality improvement system compared to conventional template-based advice or manual report creation. Application fields include support for 1-on-1 interviews in companies, communication guidance in educational settings, psychological care in medical and welfare fields, quality management in customer support, and self-improvement support services.

The provision unit can save more than ten records in the paid version. Specific functions and limitations of the paid version include, for example, the upper limit of the number of records saved, additional functions, but are not limited to such examples. The provision unit, for example, saves more than ten records in the paid version. The provision unit may also set an upper limit on the number of records saved as an additional function. By saving more than ten records in the paid version, the user can record and analyze many conversations. Some or all of the above-described processing in the provision unit may be performed using AI or without using AI. For example, the provision unit can input records to be saved to AI and have the AI manage the number of records saved. Specifically, the provision unit receives conversation record data to be saved (e.g., audio files, text-based conversation logs, JSON structures containing metadata for each statement) as input data. Examples of input data include “Jun. 1, 2024, 10:00 Conversation A audio file”, “Jun. 2, 2024, 15:30 Conversation B text log”. When storing these data in the database management system, the provision unit dynamically sets the upper limit of the number of records saved (e.g., 20, 50, etc.) and applies algorithms to automatically archive or delete old records when the limit is exceeded. When using AI, the provision unit inputs the content of the data to be saved (e.g., conversation importance score, psychological safety risk value for statements, user's emotion estimation value) to a neural network model (e.g., multilayer perceptron or decision tree-based classifier) to determine the saving priority, and generates output such as “save”, “archive”, “delete” action labels or priority scores. For example, by providing input such as “Conversation A: importance 0.92, risk 0.15”, “Conversation B: importance 0.45, risk 0.85” to AI, output such as “Conversation A: save”, “Conversation B: archive” can be obtained. As subsequent processing, the provision unit manages the upper limit of the number of records saved, automatically organizes records, and updates the saved record list on the user interface based on the AI output. As a technical effect, by using AI to dynamically manage the number of records saved and saving priority, the provision unit enables flexible and optimal record management according to each user's usage and the importance of conversation content, greatly improving the overall system's storage efficiency and user experience compared to conventional static saving limit settings or manual organization methods. Application fields include long-term storage of 1-on-1 interview records in companies, management of conversation history for each student in educational settings, record storage for each patient in medical and welfare fields, and quality management in customer support.

The provision unit can confirm the growth trend of communication skills related to psychological safety. Specific evaluation criteria and confirmation methods for growth trends include, for example, quantitative indicators, qualitative evaluations, but are not limited to such examples. The provision unit, for example, evaluates growth trends using quantitative indicators. The provision unit may also confirm growth trends using qualitative evaluations. By confirming the growth trend of communication skills related to psychological safety, the user can feel their own growth. Some or all of the above-described processing in the provision unit may be performed using AI or without using AI. For example, the provision unit can input growth trend data to AI and have the AI evaluate the growth trend. Specifically, the provision unit receives multidimensional data such as psychological safety scores extracted from each user's conversation records (e.g., risk values for each conversation, label distribution for each statement, time-series score arrays), changes in statement tendencies, and feedback history as input data. Examples of input data include time-series arrays such as “Jun. 1, 2024: risk score 0.85”, “Jun. 8, 2024: risk score 0.65”, “Jun. 15, 2024: risk score 0.42”, or “trend of aggressive statement ratio in the past ten 1-on-1 interviews”. The provision unit inputs these data to time-series analysis models (e.g., recurrent neural networks such as LSTM or GRU, or autoregressive models) or regression analysis algorithms, and generates output such as slope values for growth trends, quantitative scores for growth degree (e.g., risk score decreased by 0.4 over six months), and automatic extraction of growth points (e.g., “Significant decrease in negative statements since May”). Furthermore, AI can generate qualitative evaluations such as growth comments in natural language (e.g., “Recently, expressions that respect the other person's opinion have increased in conversations”) and summary sentences of improvement trends. Examples of output include “Growth score: +0.35”, “Aggressive statement ratio: 10%→3%”, “Growth comment: Psychological safety has greatly improved”. As subsequent processing, the provision unit visualizes these outputs as graphs (e.g., line graphs, heatmaps), displays them on the dashboard UI, and automatically incorporates them into PDF or HTML reports. As a technical effect, by using AI to automatically and accurately evaluate and visualize growth trends from multidimensional and time-series data, the provision unit enables objective and quantitative understanding of each user's growth, making continuous support for improving communication skills possible compared to conventional simple history display or subjective manual evaluation. Application fields include human resource development in companies, student guidance in educational settings, patient care in medical and welfare fields, and self-improvement support services.

The recording unit can estimate the user's emotion and adjust the timing of voice recording based on the estimated emotion of the user. For example, if the user is nervous, the recording unit may wait to start recording until the user relaxes. If the user is relaxed, the recording unit may start recording immediately. Furthermore, if the user is in a hurry, the recording unit may adjust to record the necessary information in a short time. By adjusting the timing of voice recording based on the user's emotion, voice can be recorded at a more appropriate timing. Emotion estimation is realized, for example, by using an emotion engine or generative AI with emotion estimation functions. Generative AI may include, for example, text generation AI (e.g., LLM) or multimodal generative AI, but is not limited to such examples. Some or all of the above-described processing in the recording unit may be performed using AI or without using AI. For example, the recording unit can input the user's emotion data to AI and have the AI adjust the recording timing. Specifically, the recording unit receives multimodal data such as the user's audio waveform data (e.g., 16000 samples of PCM data for 1 second), biometric sensor data (e.g., heart rate, skin conductance), and facial images (e.g., facial feature points extracted from camera images) as input data. Examples of input include “audio waveform+heart rate 90 bpm+facial expression: frown”, “audio waveform +heart rate 65 bpm+facial expression: smile”. The recording unit inputs these data to a multimodal emotion estimation model (e.g., Transformer-based neural network integrating audio, image, and biometric signals) and generates output such as emotion labels (e.g., nervous, relaxed, in a hurry) and emotion intensity scores (e.g., nervousness 0.82, relaxation 0.15). Output examples include “nervous (0.82)”, “relaxed (0.91)”, “in a hurry (0.67)”. Based on these outputs, the recording unit performs automatic control of recording start timing (e.g., wait until nervousness is below 0.7), dynamic adjustment of recording time (e.g., complete recording within 30 seconds if in a hurry), and optimization of notification timing for recording start to the user. As a technical effect, by using AI to accurately estimate the user's emotional state and dynamically optimize recording timing, the recording unit reduces the user's psychological burden and enables more natural and high-quality voice data acquisition compared to conventional uniform recording start methods. Application fields include conversation recording in counseling and coaching, patient response recording in medical and welfare fields, and interview recording in educational settings.

The recording unit can analyze the user's past conversation history and select an optimal recording method. For example, the recording unit may preferentially select a recording method that the user has preferred in the past. The recording unit may also propose a recording method suitable for specific situations based on the user's past conversation history. Furthermore, the recording unit may analyze the user's past conversation history and select the most effective recording method. By analyzing the user's past conversation history, the optimal recording method can be selected. Some or all of the above-described processing in the recording unit may be performed using AI or without using AI. For example, the recording unit can input the user's past conversation history data to AI and have the AI select the optimal recording method. Specifically, the recording unit receives conversation history data for each user (e.g., metadata of conversation records for the past year, selection history of recording methods, feature vector of conversation content) as input data. Examples of input include “May 2024: audio +text recording”, “June 2024: audio only recording”, “July 2024: recording with automatic summary”. The recording unit inputs these data to a supervised learning model (e.g., random forest, gradient boosting, or Transformer-based history analysis model) and generates output such as optimal recording method labels (e.g., audio +text, with automatic summary, audio only) and recommendation scores (e.g., audio+text 0.85, with automatic summary 0.65). Output examples include “Recommended: audio+text (0.92)”, “Recommended: with automatic summary (0.78)”. Based on these outputs, the recording unit performs automatic selection and proposal of recording methods on the user interface, automatic optimization of recording settings, and notification of recording method changes to the user. As a technical effect, by using AI to automatically select the optimal recording method from past conversation history, the recording unit enables flexible and highly efficient record management tailored to each user's usage tendencies and situations, greatly improving the convenience and recording quality of the entire system compared to conventional manual settings or uniform methods. Application fields include optimization of minute-taking methods for business meetings, proposal of recording methods for each student in educational settings, and record management for each patient in medical settings.

The recording unit can filter the user's current environmental sounds and remove noise during voice recording. For example, if the user is in a noisy environment, the recording unit filters environmental sounds and removes noise. If the user is in a quiet environment, the recording unit can perform clear voice recording. Furthermore, if the user is on the move, the recording unit can filter environmental sounds in real time and remove noise. By filtering the user's current environmental sounds and removing noise, clear voice recording becomes possible. Some or all of the above-described processing in the recording unit may be performed using AI or without using AI. For example, the recording unit can input environmental sound data to AI and have the AI perform noise removal. Specifically, the recording unit receives the user's audio signal (e.g., 16 kHz, 16 bit, 10 seconds of PCM data) and, at the same time, environmental sound data obtained from environmental sound sensors or microphone arrays (e.g., background noise waveform, spectrogram) as input data. Examples of input include “audio signal+café environment noise”, “audio signal+in-car driving noise”. The recording unit inputs these data to convolutional neural networks (CNN), U-Net, or self-supervised learning models (e.g., Denoising Autoencoder) and generates output such as noise-removed audio waveform (e.g., PCM data with improved SNR), spectral mask of noise components, and noise suppression score (e.g., SNR improvement +12 dB). Output examples include “noise-removed audio file”, “noise suppression score: +10 dB”. Based on these outputs, the recording unit performs quality evaluation of recorded audio, dynamic optimization of noise removal parameters, and feedback on recording quality to the user. As a technical effect, by using AI to achieve real-time and high-precision noise removal, the recording unit enables clear voice recording in various environments compared to conventional simple filtering or manual editing, greatly improving the recording quality and reliability of the entire conversation quality improvement system. Application fields include outdoor interview recording, conversation recording while on the move, and medical record keeping in healthcare.

The recording unit can estimate the user's emotion and determine the priority of the voice to be recorded based on the estimated emotion of the user. For example, if the user is feeling stressed, the recording unit prioritizes recording important conversations. If the user is relaxed, the recording unit may record all conversations equally. Furthermore, if the user is in a hurry, the recording unit may prioritize recording important information in a short time. By determining the priority of the voice to be recorded based on the user's emotion, important conversations can be recorded preferentially. Emotion estimation is realized, for example, by using an emotion engine or generative AI with emotion estimation functions. Generative AI may include, for example, text generation AI (e.g., LLM) or multimodal generative AI, but is not limited to such examples. Some or all of the above-described processing in the recording unit may be performed using AI or without using AI. For example, the recording unit can input the user's emotion data to AI and have the AI determine the priority of the voice to be recorded. Specifically, the recording unit simultaneously acquires multimodal data such as the user's audio waveform data (e.g., 16 kHz, 16 bit, 10 seconds of PCM data), biometric sensor data (e.g., heart rate, skin conductance), and facial images (e.g., facial feature points extracted from camera images), and inputs these to a multimodal emotion estimation model (e.g., Transformer-based neural network integrating audio, image, and biometric signals). Examples of input include “audio waveform +heart rate 100 bpm +facial expression: frown”, “audio waveform +heart rate 65 bpm +facial expression: smile”. The recording unit obtains emotion labels (e.g., stress, relaxation, in a hurry) and emotion intensity scores (e.g., stress level 0.85, relaxation level 0.12) as output from the model. Output examples include “stress (0.85)”, “relaxation (0.91)”, “in a hurry (0.67)”. The recording unit combines these emotion estimation results with conversation content importance scores (e.g., importance estimation values for conversation content by natural language processing models) and inputs them to a priority determination algorithm (e.g., multilayer perceptron or decision tree-based classifier). The priority determination algorithm uses the input emotion labels, intensity, and conversation content features to generate output such as recording action labels (e.g., “priority recording”, “normal recording”, “postponed”) and priority scores (e.g., 0.92, 0.45). For example, by providing input such as “stress (0.85) +conversation importance 0.92”, output such as “priority recording (0.95)” can be obtained. Based on these outputs, the recording unit performs reordering of the recording queue, automatic control of recording start timing, dynamic adjustment of recording time (e.g., complete recording within 30 seconds if in a hurry), and optimization of notification timing for recording start to the user. As a technical effect, by using AI to integratively evaluate the user's emotional state and conversation content importance in a high-dimensional feature space and dynamically optimize recording priority, the recording unit achieves essential improvements in computer technology, enabling high-quality recording of important conversations without omission while reducing the user's psychological burden compared to conventional uniform recording methods or manual selection. Application fields include recording of important conversations in counseling and coaching, patient response recording in medical and welfare fields, interview recording in educational settings, and creation of minutes for business meetings.

The recording unit can preferentially record highly relevant voice by considering the user's geographic location information during voice recording. For example, if the user is in a specific location, the recording unit prioritizes recording conversations related to that location. If the user is on the move, the recording unit may prioritize recording conversations related to the destination. Furthermore, if the user is participating in a specific event, the recording unit may prioritize recording conversations related to that event. By considering the user's geographic location information and preferentially recording highly relevant voice, important information can be recorded without omission. Some or all of the above-described processing in the recording unit may be performed using AI or without using AI. For example, the recording unit can input geographic location information data to AI and have the AI select highly relevant voice. Specifically, the recording unit obtains geographic location information from the user's device in real time using GPS, Wi-Fi positioning, beacon signals, etc. (e.g., latitude/longitude, facility ID, event venue name), combines it with time information and calendar information, and structures it as geographic context data. Examples of input include “Jun. 1, 2024, 10:00 Latitude 35.6895 Longitude 139.6917 (Tokyo office)”, “Jun. 2, 2024, 15:30 Event venue ID=E123”. The recording unit combines these geographic context data with conversation content metadata (e.g., conversation topic, participant information) and inputs them to a relevance estimation model (e.g., neural network integrating geographic features and conversation content features). The relevance estimation model uses the input geographic location, time, event information, and conversation content features to generate output such as “relevance score (e.g., 0.92, 0.45)” and “priority recording label (e.g., priority, normal, postponed)”. For example, by providing input such as “Tokyo office +meeting topic: project progress”, output such as “relevance score 0.91”, “priority recording” can be obtained. Based on these outputs, the recording unit performs reordering of the recording queue, automatic control of recording start timing, dynamic adjustment of recording time, and optimization of notification timing for recording start to the user. As a technical effect, by using AI to integratively evaluate the relevance of geographic location information and conversation content in a high-dimensional feature space and dynamically optimize recording priority, the recording unit achieves essential improvements in computer technology, enabling high-quality recording of important conversations for each site or event without omission compared to conventional uniform recording methods or manual selection. Application fields include optimization of field work recording, on-site conversation recording for sales activities, recording of important statements at conferences and events, and patient response recording in medical settings.

The recording unit can analyze the user's social media activity and record relevant voice during voice recording. For example, the recording unit records voice related to topics the user is discussing on social media. The recording unit may also preferentially record conversations with people the user follows on social media. Furthermore, the recording unit may analyze the user's social media activity and record voice related to relevant topics. By analyzing the user's social media activity and recording relevant voice, important conversations can be recorded without omission. Some or all of the above-described processing in the recording unit may be performed using AI or without using AI. For example, the recording unit can input social media activity data to AI and have the AI select relevant voice. Specifically, the recording unit obtains the user's social media post data (e.g., text posts, images, videos, hashtags, follow relationships, like history) via API, etc., and uses natural language processing models or graph neural networks to perform topic extraction and relationship analysis. Examples of input include “Jun. 1, 2024: #AI #conversation analysis”, “Jun. 2, 2024: Follow: Mr. A, Mr. B”, “Jun. 3, 2024: Post content ‘Discussion on psychological safety’”. The recording unit combines these social media features with real-time conversation content metadata (e.g., conversation topic, participant ID) and inputs them to a relevance estimation model (e.g., Transformer-based cross-modal relevance estimation model). The relevance estimation model uses the input social media features and conversation content features to generate output such as “relevance score (e.g., 0.88, 0.52)” and “priority recording label (e.g., priority, normal, postponed)”. For example, by providing input such as “#AI #conversation analysis +conversation topic: AI utilization”, output such as “relevance score 0.93”, “priority recording” can be obtained. Based on these outputs, the recording unit performs reordering of the recording queue, automatic control of recording start timing, dynamic adjustment of recording time, and optimization of notification timing for recording start to the user. As a technical effect, by using AI to integratively evaluate the relevance of social media activity and conversation content in a high-dimensional feature space and dynamically optimize recording priority, the recording unit achieves essential improvements in computer technology, enabling high-quality recording of important conversations tailored to the user's interests and network without omission compared to conventional uniform recording methods or manual selection. Application fields include recording of statements by influencers and PR personnel, SNS-linked recording in customer support, recording of students' topics of interest in educational settings, and understanding patients' life backgrounds in medical settings.

The conversion unit can estimate the user's emotion and adjust the conversion accuracy of the character string based on the estimated emotion of the user. For example, if the user is nervous, the conversion unit increases conversion accuracy to generate accurate character strings. If the user is relaxed, the conversion unit may maintain normal conversion accuracy. Furthermore, if the user is in a hurry, the conversion unit may prioritize conversion speed to generate character strings. By adjusting the conversion accuracy of the character string based on the user's emotion, more accurate character strings can be generated. Emotion estimation is realized, for example, by using an emotion engine or generative AI with emotion estimation functions. Generative AI may include, for example, text generation AI (e.g., LLM) or multimodal generative AI, but is not limited to such examples. Some or all of the above-described processing in the conversion unit may be performed using AI or without using AI. For example, the conversion unit can input the user's emotion data to AI and have the AI adjust conversion accuracy. Specifically, the conversion unit integrates audio tensors received from the recording unit (e.g., waveform data of 16000 samples for 1 second or spectrogram of 128 dimensions×100 frames) and the user's emotion estimation results (e.g., nervousness 0.82, relaxation 0.15, in a hurry 0.67) as input data. Examples of input include “audio waveform+nervousness 0.82”, “audio waveform+relaxation 0.91”. The conversion unit inputs these data to convolutional neural networks (CNN), recurrent neural networks (RNN), or Transformer-based speech recognition models and dynamically adjusts decoder beam width, thresholds, noise suppression parameters, etc., according to the emotional state. For example, if nervousness is high, the beam search width is widened to increase the number of candidates and reduce the risk of misrecognition. Conversely, if in a hurry, the decoder search width is narrowed to prioritize speed. Output includes UTF-8 encoded character strings (e.g., “Thank you for today”), conversion confidence score (e.g., 0.98), and conversion speed (e.g., 0.5 seconds/sentence). As subsequent processing, the conversion unit passes the output character string and confidence score to the analysis unit for psychological safety evaluation and analysis of statement tendencies. As a technical effect, by using AI to dynamically optimize the parameters of the speech recognition model according to the user's emotional state, the conversion unit enables high-precision and high-efficiency character string generation tailored to the user experience and situation, greatly improving the reliability and practicality of the entire conversation quality improvement system compared to conventional uniform conversion accuracy settings or manual adjustment methods. Application fields include creation of minutes for business meetings, lesson recording in educational settings, medical record keeping in healthcare, and conversation recording in counseling settings.

The conversion unit can improve conversion accuracy by considering the context of the conversation during voice conversion. For example, the conversion unit analyzes the preceding and following context of the conversation and selects appropriate words to improve conversion accuracy. The conversion unit may also improve the conversion accuracy of technical terms and proper nouns based on the topic of the conversation. Furthermore, the conversion unit may consider the flow of the conversation to convert into natural sentences. By considering the context of the conversation to improve conversion accuracy, more natural sentences can be generated. Some or all of the above-described processing in the conversion unit may be performed using AI or without using AI. For example, the conversion unit can input conversation context data to AI and have the AI improve conversion accuracy. Specifically, the conversion unit integrates audio tensors received from the recording unit (e.g., waveform data of 16000 samples for 1 second or spectrogram of 128 dimensions×100 frames) and text data of conversation history (e.g., character string sequence of the previous five sentences or topic label array) as input data. Examples of input include “audio waveform+previous sentence: ‘Today we will talk about the progress of the meeting’”, “audio waveform+topic: ‘project management’”. The conversion unit inputs these data to Transformer-based speech recognition models or encoder-decoder type large-scale language models and integratively processes acoustic features and contextual features using multi-layer attention mechanisms. The conversion unit dynamically selects decoder beam search width and pre-trained topic embeddings of the language model, and contextually corrects candidate scores for technical terms and proper nouns. Output includes UTF-8 encoded character strings (e.g., “Today we will talk about the progress of the meeting”), conversion confidence score (e.g., 0.98), and context fit score (e.g., 0.93). For example, by providing input such as “audio waveform +previous sentence: ‘Latest trends in AI technology’”, output such as “I will explain the latest trends in AI technology” or “Today we will discuss the evolution of AI technology” can be obtained as contextually appropriate natural sentences. The conversion unit passes these outputs to the analysis unit for psychological safety evaluation and analysis of statement tendencies. As subsequent processing, the conversion unit may perform re-conversion or candidate suggestion when context fit is low and provide an interface for user selection. As a technical effect, by using AI to integratively analyze conversation context in a multidimensional feature space and dynamically optimize the parameters of the speech recognition model decoder and language model, the conversion unit greatly improves recognition accuracy for context dependence, technical terms, and proper nouns compared to conventional simple sequential conversion or dictionary matching methods, dramatically enhancing the natural language generation capability and user experience of the entire conversation quality improvement system. Application fields include creation of minutes for business meetings, interview recording in specialized fields, medical record keeping in healthcare, lesson recording in educational settings, and conversation recording in counseling settings.

The conversion unit can apply conversion algorithms corresponding to different languages and dialects during voice conversion. For example, if the user speaks in a different language, the conversion unit applies a conversion algorithm corresponding to that language. If the user uses a dialect, the conversion unit may apply a conversion algorithm corresponding to that dialect. Furthermore, if the user mixes multiple languages, the conversion unit may apply conversion algorithms corresponding to each language. By applying conversion algorithms corresponding to different languages and dialects, character strings corresponding to various languages and dialects can be generated. Some or all of the above-described processing in the conversion unit may be performed using AI or without using AI. For example, the conversion unit can input audio data of different languages and dialects to AI and have the AI apply conversion algorithms. Specifically, the conversion unit integrates audio tensors received from the recording unit (e.g., waveform data of 16000 samples for 1 second or spectrogram of 128 dimensions×100 frames) and metadata for language/dialect identification (e.g., language ID, dialect ID, speaker profile) as input data. Examples of input include “audio waveform +language ID: Japanese”, “audio waveform+dialect ID: Kansai dialect”, “audio waveform+language ID: English+Japanese mixed”. The conversion unit inputs these data to multilingual Transformer-based speech recognition models or language identification models (e.g., XLSR, Wav2Vec2.0 multilingual model), and dynamically switches appropriate decoders and language models based on acoustic features and identification results. The conversion unit selects optimized acoustic models, vocabulary dictionaries, and pronunciation dictionaries for each language/dialect, and adjusts parameters of beam search and CTC decoders. Output includes UTF-8 encoded character strings (e.g., “Thank you very much”, “ [symbol] [symbol] ”), language/dialect labels (e.g., Japanese-Kansai dialect, English), and conversion confidence score (e.g., 0.97). For example, by providing input such as “audio waveform+language ID: English+Japanese mixed”, output such as “Thank you for your help. [symbol] ” can be obtained as a code-switching character string. The conversion unit passes these outputs to the analysis unit for psychological safety evaluation and analysis of statement tendencies. As subsequent processing, the conversion unit may perform dynamic learning of speaker adaptation and pronunciation variants for each user to reduce misrecognition risk for each language/dialect. As a technical effect, by using AI to dynamically optimize multilingual and multidialect speech recognition models, the conversion unit greatly improves recognition accuracy and flexibility for global usage environments and diverse speakers and speech patterns compared to conventional single-language and non-dialect models, dramatically enhancing the practicality and accessibility of the entire conversation quality improvement system. Application fields include creation of minutes for international conferences, multilingual customer support, multicultural lesson recording in educational settings, multilingual medical record keeping in healthcare, and regional dialect preservation projects.

The conversion unit can estimate the user's emotion and adjust the length of the character string to be converted based on the estimated emotion of the user. For example, if the user is in a hurry, the conversion unit converts to a short character string that captures the main points. If the user is relaxed, the conversion unit may convert to a longer character string that includes detailed explanations. Furthermore, if the user is excited, the conversion unit may convert to a character string that reflects emotion. By adjusting the length of the character string to be converted based on the user's emotion, character strings of appropriate length can be generated. Emotion estimation is realized, for example, by using an emotion engine or generative AI with emotion estimation functions. Generative AI may include, for example, text generation AI (e.g., LLM) or multimodal generative AI, but is not limited to such examples. Some or all of the above-described processing in the conversion unit may be performed using AI or without using AI. For example, the conversion unit can input the user's emotion data to AI and have the AI adjust the length of the character string. Specifically, the conversion unit integrates audio tensors received from the recording unit (e.g., waveform data of 16000 samples for 1 second or spectrogram of 128 dimensions×100 frames) and emotion estimation results (e.g., in a hurry 0.85, relaxation 0.91, excitement 0.78) as input data. Examples of input include “audio waveform+in a hurry 0.85”, “audio waveform+relaxation 0.91”, “audio waveform+excitement 0.78”. The conversion unit inputs these data to Transformer-based speech recognition models or large-scale language models and dynamically adjusts decoder output length control parameters (e.g., maximum output length, summarization rate, detail rate) and generation temperature according to the emotional state. For example, if in a hurry, the summarization rate is increased to prioritize short sentence generation; if relaxed, the detail rate is increased to generate explanatory long sentences; if excited, emotion emphasis tokens are inserted to generate sentences with stronger emotional expression. Output includes UTF-8 encoded character strings (e.g., “Main point only: The meeting starts at 3 PM”, “Detailed explanation: Today's meeting starts at 3 PM, and the agenda is . . . ”), output length (e.g., 15 characters, 120 characters), and emotion reflection score (e.g., 0.88). For example, by providing input such as “audio waveform +in a hurry 0.85”, output such as “The meeting starts at 3 PM” can be obtained as a short sentence, and with “audio waveform +relaxation 0.91”, output such as “Today's meeting starts at 3 PM, and the agenda is . . . ” can be obtained as a detailed sentence. The conversion unit passes these outputs to the analysis unit for psychological safety evaluation and analysis of statement tendencies. As subsequent processing, the conversion unit may automatically optimize and personalize output length control parameters based on user feedback and usage history. As a technical effect, by using AI to integratively analyze the user's emotional state and voice content in a high-dimensional feature space and dynamically optimize the length and emotion reflection of character string generation, the conversion unit enables flexible and high-precision character string generation tailored to the user experience and situation, greatly improving the practicality and user satisfaction of the entire conversation quality improvement system compared to conventional uniform output length settings or manual summarization methods. Application fields include creation of summary minutes for business meetings, detailed lesson recording in educational settings, medical summary record keeping in healthcare, and emotion-reflective recording in counseling settings.

The conversion unit can determine the priority of conversion based on the importance of the conversation during voice conversion. For example, the conversion unit prioritizes the conversion of important conversations and quickly generates character strings. The conversion unit may also convert general conversations with normal priority. Furthermore, the conversion unit may postpone the conversion of conversations with low importance. By determining the conversion priority based on the importance of the conversation, important conversations can be quickly converted into character strings. Some or all of the above-described processing in the conversion unit may be performed using AI, or may be performed without using AI. For example, the conversion unit can input conversation importance data into AI and have the AI determine the conversion priority. Specifically, the conversion unit integrates input data such as voice tensors received from the recording unit (e.g., 16,000 waveform samples per second or a spectrogram of 128 dimensions×100 frames) and conversation importance scores (e.g., importance estimates of 0.92, 0.45 by a natural language processing model). Examples of input include “voice waveform+importance 0.92” and “voice waveform+importance 0.45”. The conversion unit inputs these data into a multilayer perceptron, a decision tree-based classifier, or a Transformer-based priority determination model, and dynamically controls the reordering of the conversion queue and the allocation of simultaneous parallel conversion processing. For conversations with high importance, the conversion unit prioritizes fast conversion algorithms and resource allocation, while for conversations with low importance, batch processing or deferred conversion is applied. Outputs include UTF-8 encoded character strings (e.g., “Today is an important meeting”), conversion priority labels (e.g., priority, normal, deferred), and conversion speed (e.g., 0.3 seconds per sentence). For example, when “voice waveform+importance 0.92” is given as input, the output may be “priority conversion: Today is an important meeting”. The conversion unit passes these outputs to the analysis unit for use in psychological safety evaluation and statement trend analysis. In subsequent processing, the conversion unit can also optimize progress display and conversion completion notifications on the user interface according to conversion priority. As a technical effect, by using AI to quantitatively evaluate the importance of conversations in a high-dimensional feature space and dynamically optimize conversion processing priority and resource allocation, the conversion unit greatly improves the speed of converting important conversations into character strings and the overall system processing efficiency compared to conventional uniform conversion order or manual allocation methods, thereby dramatically enhancing user experience and business efficiency. Application fields include the creation of important minutes for business meetings, emergency medical records in medical settings, recording of important statements in educational settings, and prioritized conversation recording in counseling settings.

The conversion unit can adjust the order of conversion based on the relevance of the conversation during voice conversion. For example, the conversion unit prioritizes the conversion of highly relevant conversations. The conversion unit may also postpone the conversion of conversations with low relevance. Furthermore, the conversion unit can consider the flow of the conversation and convert highly relevant parts first. By adjusting the conversion order based on the relevance of the conversation, important conversations can be preferentially converted into character strings. Some or all of the above-described processing in the conversion unit may be performed using AI, or may be performed without using AI. For example, the conversion unit can input conversation relevance data into AI and have the AI adjust the conversion order. Specifically, the conversion unit integrates input data such as voice tensors received from the recording unit (e.g., 16,000 waveform samples per second or a spectrogram of 128 dimensions ×100 frames) and conversation relevance scores (e.g., relevance estimates of 0.88, 0.52 by a natural language processing model). Examples of input include “voice waveform+relevance 0.88” and “voice waveform+relevance 0.52”. The conversion unit inputs these data into a Transformer-based relevance estimation model or a multilayer perceptron, and dynamically controls the order of the conversion queue and the allocation of simultaneous parallel conversion processing. For conversations with high relevance, the conversion unit preferentially allocates conversion processing, while for conversations with low relevance, batch processing or deferred conversion is applied. Outputs include UTF-8 encoded character strings (e.g., “Project progress report”), conversion order labels (e.g., priority, normal, deferred), and relevance scores (e.g., 0.88). For example, when “voice waveform+relevance 0.88” is given as input, the output may be “priority conversion: Project progress report”. The conversion unit passes these outputs to the analysis unit for use in psychological safety evaluation and statement trend analysis. In subsequent processing, the conversion unit can also optimize conversion progress display and conversion completion notifications on the user interface according to relevance scores. As a technical effect, by using AI to quantitatively evaluate the relevance of conversations in a high-dimensional feature space and dynamically optimize the order of conversion processing and resource allocation, the conversion unit greatly improves the speed of converting important conversations into character strings and the overall system processing efficiency compared to conventional uniform conversion order or manual allocation methods, thereby dramatically enhancing user experience and business efficiency. Application fields include optimization of field work records, recording of on-site conversations in sales activities, recording of important statements at academic conferences and events, and recording of patient interactions in medical settings.

The analysis unit can estimate the user's emotion and adjust the criteria for analysis based on the estimated emotion of the user. For example, when the user is nervous, the analysis unit performs analysis with strict criteria. The analysis unit may also perform analysis with normal criteria when the user is relaxed. Furthermore, when the user is in a hurry, the analysis unit may relax the criteria to perform rapid analysis. By adjusting the criteria for analysis based on the user's emotion, more appropriate analysis becomes possible. Emotion estimation is realized, for example, by using an emotion engine or generative AI with emotion estimation functions. Generative AI may be, for example, a text generation AI (such as LLM) or a multimodal generative AI, but is not limited to these examples. Some or all of the above-described processing in the analysis unit may be performed using AI, or may be performed without using AI. For example, the analysis unit can input the user's emotion data into AI and have the AI adjust the analysis criteria. Specifically, the analysis unit inputs multimodal data such as the user's voice waveform data received from the recording unit or conversion unit (e.g., 16,000 PCM samples per second), biometric sensor data (e.g., heart rate, skin conductance), and facial images (e.g., facial feature points extracted from camera images) into an emotion estimation model (e.g., a Transformer-based neural network integrating voice, image, and biometric signals), and obtains outputs such as emotion labels (e.g., nervous, relaxed, in a hurry) and emotion intensity scores (e.g., nervousness 0.82, relaxation 0.15). Examples of input include “voice waveform+heart rate 90 bpm+facial expression: frown” and “voice waveform+heart rate 65 bpm+facial expression: smile”. The analysis unit combines these emotion estimation results with character string data received from the conversion unit (e.g., “You are useless”, “Thank you for today”, etc.), and inputs them into an analysis criteria adjustment algorithm (e.g., rule-based judgment with threshold control, large language model with emotion-dependent weighting). The analysis unit dynamically adjusts the analysis criteria, such as tightening the threshold for psychological safety risk when nervousness is high, applying normal thresholds when relaxation is high, and narrowing down analysis items for speed when in a hurry. Outputs include labels for each statement (e.g., 0=safe, 1=aggressive, 2=negative), risk scores (e.g., 0.85), and analysis criteria application labels (e.g., strict, normal, relaxed). For example, when “nervous (0.82)+statement: ‘Can't you even do that?’” is given as input, the output may be “aggressive (score 0.92, criteria: strict)”. In subsequent processing, the analysis unit generates feedback for each statement, creates reports, and displays heatmaps based on the output labels and scores. As a technical effect, by using AI to dynamically optimize the analysis criteria in a high-dimensional feature space according to the user's emotional state, the analysis unit enables flexible and highly accurate statement evaluation tailored to the user experience and situation, greatly improving the reliability and usefulness of the overall conversation quality improvement system compared to conventional uniform analysis criteria or manual adjustment methods. Application fields include conversation analysis in counseling and coaching settings, patient interaction records in medical and welfare fields, interview records in educational settings, and minutes analysis in business meetings.

The analysis unit can improve the accuracy of analysis by considering the interrelationship of conversations during analysis. For example, the analysis unit analyzes the context before and after the conversation to accurately grasp the intent of statements. The analysis unit may also analyze the impact of statements by considering the relationships among participants in the conversation. Furthermore, the analysis unit may evaluate the importance of statements by considering the flow of the conversation. By improving the accuracy of analysis by considering the interrelationship of conversations, the intent of statements can be accurately grasped. Some or all of the above-described processing in the analysis unit may be performed using AI, or may be performed without using AI. For example, the analysis unit can input conversation interrelationship data into AI and have the AI improve analysis accuracy. Specifically, the analysis unit integrates input data such as text data of conversation history received from the conversion unit (e.g., a sequence of the last 10 sentences, a list of statements with speaker IDs, time series information between statements) and relationship data among conversation participants (e.g., intimacy scores between speakers, history of past interactions). Examples of input include “Statement 1: A ‘Today we will talk about the progress of the meeting’”, “Statement 2: B ‘Thank you’”, “Statement 3: A ‘Let's start with the progress report’”, and “A-B intimacy 0.85”. The analysis unit inputs these data into a Transformer-based context analysis model or a graph neural network, and analyzes dependencies between statements and the flow of conversation using a multi-layer attention mechanism. The analysis unit outputs intent estimation for each statement (e.g., question, instruction, agreement, denial), impact scores (e.g., 0.92), and importance labels (e.g., high, medium, low). For example, when “Statement 1: A ‘What do you think about this?’+Statement 2: B ‘I agree’” is given as input, the output may be “Statement 1: question (high importance)”, “Statement 2: agreement (medium importance)”. In subsequent processing, the analysis unit generates feedback for each statement, creates reports, and visualizes the overall flow of the conversation based on the output intent, impact, and importance. As a technical effect, by using AI to integratively analyze the interrelationship of conversations in a high-dimensional feature space and dynamically evaluate statement intent, impact, and importance, the analysis unit enables highly accurate conversation analysis that reflects context dependency and participant relationships, greatly enhancing the practicality and user experience of the overall conversation quality improvement system compared to conventional simple sequential analysis or keyword extraction methods. Application fields include minutes analysis of business meetings, group discussion analysis in educational settings, multi-professional collaboration records in medical settings, and intent understanding in counseling settings.

The analysis unit can perform analysis by considering attribute information of conversation participants during analysis. For example, the analysis unit analyzes the impact of statements by considering the age and gender of conversation participants. The analysis unit may also evaluate the importance of statements by considering the occupation and position of conversation participants. Furthermore, the analysis unit may accurately grasp the intent of statements by considering the relationships among conversation participants. By performing analysis by considering attribute information of conversation participants, the impact of statements can be accurately evaluated. Some or all of the above-described processing in the analysis unit may be performed using AI, or may be performed without using AI. For example, the analysis unit can input participant attribute information data into AI and have the AI perform analysis. Specifically, the analysis unit integrates input data such as attribute data for each conversation participant (e.g., age, gender, occupation, position, department, past statement trend scores) and statement text received from the conversion unit (e.g., “Thank you for today”, “I will be in charge of this matter”, etc.). Examples of input include “Participant A: age 35, male, position: manager”, “Participant B: age 28, female, position: staff”, and “Statement: A ‘Please give a progress report’”. The analysis unit inputs these data into a large language model with attribute information embedding or an attribute-conditioned classifier (e.g., multilayer perceptron, decision tree), and outputs statement impact scores (e.g., 0.92), importance labels (e.g., high, medium, low), and intent estimation (e.g., instruction, request, agreement). For example, when “Statement: A (manager) ‘Please make sure to handle this matter’+B (staff)” is given as input, the output may be “High impact (0.95), intent: instruction, importance: high”. In subsequent processing, the analysis unit generates feedback for each statement, creates reports, and performs trend analysis by attribute based on the output impact, importance, and intent. As a technical effect, by using AI to integratively analyze attribute information of conversation participants in a high-dimensional feature space and dynamically evaluate statement impact, importance, and intent, the analysis unit enables highly accurate conversation analysis that reflects the roles and relationships of each participant, greatly enhancing the practicality and user experience of the overall conversation quality improvement system compared to conventional uniform statement evaluation or attribute-agnostic methods. Application fields include hierarchical meeting analysis in companies, communication analysis between students and teachers in educational settings, multi-professional collaboration records in medical settings, and trend analysis of statements by attribute in counseling settings.

The analysis unit can estimate the user's emotion and adjust the display order of analysis results based on the estimated emotion of the user. For example, when the user is nervous, the analysis unit displays important analysis results first. The analysis unit may also display all analysis results equally when the user is relaxed. Furthermore, when the user is in a hurry, the analysis unit may display key analysis results first. By adjusting the display order of analysis results based on the user's emotion, important analysis results can be preferentially displayed. Emotion estimation is realized, for example, by using an emotion engine or generative AI with emotion estimation functions. Generative AI may be, for example, a text generation AI (such as LLM) or a multimodal generative AI, but is not limited to these examples. Some or all of the above-described processing in the analysis unit may be performed using AI, or may be performed without using AI. For example, the analysis unit can input the user's emotion data into AI and have the AI adjust the display order. Specifically, the analysis unit inputs multimodal data such as the user's voice waveform data, biometric sensor data, and facial images received from the recording unit or conversion unit into an emotion estimation model, and obtains emotion labels (e.g., nervous, relaxed, in a hurry) and emotion intensity scores (e.g., nervousness 0.82). Examples of input include “voice waveform+nervousness 0.82” and “voice waveform+relaxation 0.91”. The analysis unit combines these emotion estimation results with analysis results for each statement generated by the analysis unit (e.g., statement labels, risk scores, importance scores), and inputs them into a display order determination algorithm (e.g., multilayer perceptron or decision tree-based classifier). The analysis unit dynamically adjusts the display order, such as placing analysis results with high importance scores at the top when nervousness is high, displaying all analysis results equally when relaxation is high, and prioritizing only key points when in a hurry. Outputs include analysis result lists with display order (e.g., 1st: high importance, 2nd: key point, 3rd: details), display order labels (e.g., priority, normal, deferred), etc. For example, when “nervous (0.82)+analysis result list” is given as input, the output may be “1st: aggressive statement (score 0.92), 2nd: negative statement (score 0.75)”. In subsequent processing, the analysis unit passes the output analysis results with display order to the provision unit, and optimizes the display order and notification timing on the user interface. As a technical effect, by using AI to integratively evaluate the user's emotional state and the importance of analysis results in a high-dimensional feature space and dynamically optimize the display order, the analysis unit enables flexible and highly efficient information presentation tailored to the user experience and situation, greatly improving the practicality and user satisfaction of the overall conversation quality improvement system compared to conventional uniform display order or manual sorting methods. Application fields include presentation of important analysis results in counseling and coaching settings, patient interaction records in medical and welfare fields, interview records in educational settings, and minutes analysis in business meetings.

The analysis unit can perform analysis by considering the geographical distribution of conversations during analysis. For example, the analysis unit analyzes the impact of statements by considering the location where the conversation took place. The analysis unit may also analyze the geographical distribution of conversations to grasp the trends of statements in each region. Furthermore, the analysis unit may evaluate the importance of statements based on the location of the conversation. By performing analysis by considering the geographical distribution of conversations, trends of statements in each region can be grasped. Some or all of the above-described processing in the analysis unit may be performed using AI, or may be performed without using AI. For example, the analysis unit can input geographical distribution data into AI and have the AI perform analysis. Specifically, the analysis unit integrates input data such as geographical location information for each conversation received from the recording unit or conversion unit (e.g., GPS coordinates, facility ID, event venue name, city name), time information, and text data of conversation content (e.g., “Today is a meeting in Osaka”, “Progress report on field work”, etc.). Examples of input include “Jun. 1, 2024, 10:00, latitude 35.6895, longitude 139.6917 (Tokyo office)+statement: ‘Project progress’”, “Jun. 2, 2024, 15:30, Osaka venue+statement: ‘Field work’”. The analysis unit inputs these geographical context data and conversation content features into a large language model with geographical information embedding or a geographical clustering algorithm (e.g., K-means, DBSCAN), and outputs regional statement trend scores (e.g., 0.92), impact labels (e.g., high, medium, low), and importance scores (e.g., 0.85). For example, when “Osaka venue+statement: ‘Field work’” is given as input, the output may be “Regional trend: many field work statements (score 0.91), importance: high”. In subsequent processing, the analysis unit generates feedback for each statement, creates reports, and displays regional heatmaps based on the output regional trends, impact, and importance. As a technical effect, by using AI to integratively analyze the geographical distribution and content of conversations in a high-dimensional feature space and dynamically evaluate regional statement trends, impact, and importance, the analysis unit enables highly accurate conversation analysis that reflects the characteristics of each site or region, greatly enhancing the practicality and user experience of the overall conversation quality improvement system compared to conventional uniform statement evaluation or methods that do not consider geographical information. Application fields include optimization of field work records, recording of on-site conversations in sales activities, recording of important statements at academic conferences and events, and regional patient interaction records in medical settings.

The analysis unit can improve the accuracy of analysis by referring to related literature during analysis. For example, the analysis unit refers to literature related to the content of the conversation to accurately grasp the intent of statements. The analysis unit may also analyze the impact of statements by referring to research results related to the topic of the conversation. Furthermore, the analysis unit may evaluate the importance of statements based on related literature while considering the flow of the conversation. By improving the accuracy of analysis by referring to related literature, the intent of statements can be accurately grasped. Some or all of the above-described processing in the analysis unit may be performed using AI, or may be performed without using AI. For example, the analysis unit can input related literature data into AI and have the AI improve analysis accuracy. Specifically, the analysis unit integrates input data such as text data of conversation content received from the conversion unit (e.g., “Discussion on psychological safety”, “Latest trends in AI technology”, etc.) and related literature databases (e.g., paper titles, abstracts, keywords, DOI, publication year). Examples of input include “Conversation topic: psychological safety+literature: ‘Theory and Practice of Psychological Safety’”, “Conversation topic: AI utilization+literature: ‘Latest Research on Conversation Analysis by AI’”. The analysis unit inputs these data into a large language model with literature search or a knowledge graph-based relevance estimation model, and outputs similarity scores between conversation content and literature content (e.g., 0.92), statement intent estimation (e.g., theory reference, presentation of practical examples), and impact/importance labels (e.g., high, medium, low). For example, when “Conversation topic: psychological safety+literature: ‘Theory and Practice of Psychological Safety’” is given as input, the output may be “Intent: theory reference (score 0.91), importance: high”. In subsequent processing, the analysis unit generates feedback for each statement, creates reports, and presents related literature lists based on the output intent, impact, and importance. As a technical effect, by using AI to integratively analyze conversation content and related literature in a high-dimensional feature space and dynamically evaluate statement intent, impact, and importance, the analysis unit enables highly accurate conversation analysis that reflects theoretical grounds and the latest research, greatly improving the reliability and academic value of the overall conversation quality improvement system compared to conventional methods that do not refer to literature or manual search methods. Application fields include minutes analysis of academic conferences, research presentation records in educational settings, evidence-based medical records in medical settings, and market research reports in business meetings.

The provision unit can estimate the user's emotion and adjust the display method of the report based on the estimated emotion of the user. For example, when the user is nervous, the provision unit provides a simple and highly visible report. The provision unit may also provide a report containing detailed information when the user is relaxed. Furthermore, when the user is in a hurry, the provision unit may provide a report that emphasizes key points. By adjusting the display method of the report based on the user's emotion, more appropriate reports can be provided. Emotion estimation is realized, for example, by using an emotion engine or generative AI with emotion estimation functions. Generative AI may be, for example, a text generation AI (such as LLM) or a multimodal generative AI, but is not limited to these examples. Some or all of the above-described processing in the provision unit may be performed using AI, or may be performed without using AI. For example, the provision unit can input the user's emotion data into AI and have the AI adjust the display method of the report. Specifically, the provision unit inputs multimodal data such as the user's voice waveform data (e.g., 16,000 PCM samples per second), biometric sensor data (e.g., heart rate, skin conductance), and facial images (e.g., facial feature points extracted from camera images) received from the recording unit or analysis unit into an emotion estimation model (e.g., a Transformer-based neural network integrating voice, image, and biometric signals), and obtains outputs such as emotion labels (e.g., nervous, relaxed, in a hurry) and emotion intensity scores (e.g., nervousness 0.82, relaxation 0.15). Examples of input include “voice waveform+heart rate 90 bpm+facial expression: frown” and “voice waveform+heart rate 65 bpm+facial expression: smile”. The provision unit combines these emotion estimation results with report generation data received from the analysis unit (e.g., labels for each statement, risk scores, importance scores, time series data), and inputs them into a display method determination algorithm (e.g., multilayer perceptron or decision tree-based classifier, or a large language model with template selection). The provision unit automatically selects a simple layout, large font, and design with fewer colors when nervousness is high, generates a rich report with detailed graphs, annotations, and past comparison charts when relaxation is high, and generates a summary report emphasizing only key points when in a hurry. Outputs include report files in PDF, HTML, or dashboard UI formats, display template IDs, and display element lists (e.g., graphs, text, key point lists). For example, when “nervous (0.82)+analysis result list” is given as input, the output may be “simple report (key points only, large font)” or “detailed report (graph+annotation)”. In subsequent processing, the provision unit displays the output report in the optimal format on the user interface and collects user browsing history and feedback to reflect in the optimization of display methods for future reports. As a technical effect, by using AI to integratively analyze the user's emotional state and report content in a high-dimensional feature space and dynamically optimize the report display method, the provision unit enables flexible and highly efficient information presentation tailored to the user experience and situation, greatly improving the practicality and user satisfaction of the overall conversation quality improvement system compared to conventional uniform report display or manual switching methods. Application fields include presentation of important analysis results in counseling and coaching settings, patient interaction records in medical and welfare fields, interview records in educational settings, and minutes analysis in business meetings.

The provision unit can optimize the current report by referring to past report data when providing the report. For example, the provision unit refers to the user's past report data and reflects it in the current report. The provision unit may also analyze past report data to improve the accuracy of the current report. Furthermore, the provision unit may propose the optimal report format based on the user's past report data. By optimizing the current report by referring to past report data, more accurate reports can be provided. Some or all of the above-described processing in the provision unit may be performed using AI, or may be performed without using AI. For example, the provision unit can input past report data into AI and have the AI optimize the report. Specifically, the provision unit obtains past report data for each user (e.g., PDF files, HTML reports, structured data in JSON format, report browsing history, user feedback, report format selection history) from a database and inputs them into a report optimization model (e.g., a large language model with history analysis or a template recommendation algorithm). Examples of input include “Jun. 1, 2024: PDF report (key point emphasis type)”, “Jun. 8, 2024: HTML report (detailed type)”, “Jun. 15, 2024: user feedback: graph is easy to see”. The provision unit analyzes report content trends (e.g., points emphasized in the past, frequently referenced graphs), extracts user preferences and browsing patterns, and optimizes report formats (e.g., key point type, detailed type, graph-focused type). Outputs include optimized report template IDs, display element lists (e.g., graphs, key point lists, annotations), and automatic summary or detail parameters for report content. For example, when “high graph viewing rate in the past three reports+key point type is preferred” is given as input, the output may be “graph-focused key point report”. In subsequent processing, the provision unit automatically generates the optimized report and presents it to the user, while continuously collecting new feedback and browsing history to reflect in the report optimization algorithm for future reports. As a technical effect, by using AI to integratively analyze past report data and user behavior history in a high-dimensional feature space and dynamically optimize report content and format, the provision unit enables highly accurate report generation and continuous quality improvement tailored to each user's needs and usage trends, greatly improving the usefulness and user satisfaction of the overall conversation quality improvement system compared to conventional static template methods or manual customization methods. Application fields include support for 1-on-1 meetings in companies, individual instruction records in educational settings, patient progress reports in medical and welfare fields, and quality management in customer support.

The provision unit can apply different report formats for each category of conversation when providing the report. For example, in the case of business conversations, the provision unit provides a formal report format. The provision unit may also provide a friendly report format for casual conversations. Furthermore, for academic conversations, the provision unit may provide a report format that includes detailed analysis. By applying different report formats for each category of conversation, more appropriate reports can be provided. Some or all of the above-described processing in the provision unit may be performed using AI, or may be performed without using AI. For example, the provision unit can input conversation category data into AI and have the AI apply the report format. Specifically, the provision unit receives input data such as conversation category data from the analysis unit or conversion unit (e.g., category labels such as business, casual, academic, medical, educational, conversation topics, participant attributes, conversation content features). Examples of input include “Category: business+topic: project progress”, “Category: casual+topic: chat”, “Category: academic+topic: AI technology”. The provision unit inputs these data into a category-conditioned report generation model (e.g., a large language model with template selection or a category classifier), and automatically selects optimized report templates and display elements for each category (e.g., formal layout, friendly design, detailed analysis graphs). Outputs include report template IDs, display element lists, and category fit scores (e.g., 0.95). For example, when “Category: business+topic: project progress” is given as input, the output may be “formal report (key points+progress graph)”. In subsequent processing, the provision unit automatically generates the optimized report for each category and presents it to the user. Furthermore, the provision unit can collect user feedback and browsing history for each category and reflect them in the report format optimization algorithm for future reports. As a technical effect, by using AI to integratively analyze conversation categories and report formats in a high-dimensional feature space and dynamically generate optimized reports for each category, the provision unit enables highly accurate information presentation and improved user satisfaction tailored to the usage scene and purpose, greatly improving the practicality of the overall conversation quality improvement system compared to conventional uniform report formats or manual switching methods. Application fields include minutes creation for business meetings, lesson records in educational settings, presentation records at academic conferences, medical records in medical settings, and support for casual communication.

The provision unit can estimate the user's emotion and adjust the importance of the report based on the estimated emotion of the user. For example, when the user is nervous, the provision unit provides a report that emphasizes important points. The provision unit may also provide overall information equally when the user is relaxed. Furthermore, when the user is in a hurry, the provision unit may provide a report that concisely summarizes key points. By adjusting the importance of the report based on the user's emotion, a report that emphasizes important points can be provided. Emotion estimation is realized, for example, by using an emotion engine or generative AI with emotion estimation functions. Generative AI may be, for example, a text generation AI (such as LLM) or a multimodal generative AI, but is not limited to these examples. Some or all of the above-described processing in the provision unit may be performed using AI, or may be performed without using AI. For example, the provision unit can input the user's emotion data into AI and have the AI adjust the importance of the report. Specifically, the provision unit inputs multimodal data such as the user's voice waveform data, biometric sensor data, and facial images received from the recording unit or analysis unit into an emotion estimation model, and obtains emotion labels (e.g., nervous, relaxed, in a hurry) and emotion intensity scores (e.g., nervousness 0.82). Examples of input include “voice waveform+nervousness 0.82” and “voice waveform+relaxation 0.91”. The provision unit combines these emotion estimation results with report generation data received from the analysis unit (e.g., importance scores for each statement, risk scores, key point lists), and inputs them into an importance adjustment algorithm (e.g., weighted template engine or large language model with importance emphasis). The provision unit emphasizes important points when nervousness is high, arranges overall information equally when relaxation is high, and generates a report that concisely summarizes only key points when in a hurry. Outputs include report files with emphasis elements, importance label lists, and emphasis scores (e.g., 0.95). For example, when “nervous (0.82)+key point list” is given as input, the output may be “key point emphasis report (important points displayed in red)”. In subsequent processing, the provision unit displays the generated report in the optimal format on the user interface and collects user browsing history and feedback to reflect in the importance adjustment algorithm for future reports. As a technical effect, by using AI to integratively analyze the user's emotional state and report content in a high-dimensional feature space and dynamically optimize the importance of the report, the provision unit enables flexible and highly efficient information presentation tailored to the user experience and situation, greatly improving the practicality and user satisfaction of the overall conversation quality improvement system compared to conventional uniform emphasis methods or manual editing methods. Application fields include presentation of important analysis results in counseling and coaching settings, patient interaction records in medical and welfare fields, interview records in educational settings, and minutes analysis in business meetings.

The provision unit can determine the priority of the report based on the submission timing of the conversation when providing the report. For example, the provision unit preferentially provides reports for recent conversations. The provision unit may also provide reports for past conversations with normal priority. Furthermore, the provision unit may preferentially provide reports for conversations related to specific events immediately after the event. By determining the priority of the report based on the submission timing of the conversation, important conversations can be quickly provided as reports. Some or all of the above-described processing in the provision unit may be performed using AI, or may be performed without using AI. For example, the provision unit can input submission timing data into AI and have the AI determine the priority of the report. Specifically, the provision unit receives input data such as submission time data for conversation records (e.g., timestamp, event ID, conversation type, conversation content features). Examples of input include “Jun. 1, 2024, 10:00, Conversation A”, “Jun. 2, 2024, 15:30, Conversation B (event ID=E 123)”. The provision unit inputs these data into a time series priority determination model (e.g., recurrent neural network or decision tree-based classifier), and automatically determines the reordering of the report generation queue and priority labels (e.g., priority, normal, deferred) based on submission timing and event information. Outputs include report lists with priority, priority labels, and generation order scores (e.g., 0.95). For example, when “Conversation A (latest)”, “Conversation B (immediately after event)” are given as input, the output may be “Conversation A: priority”, “Conversation B: priority”. In subsequent processing, the provision unit optimizes the order of report generation and delivery according to priority, and automatically adjusts notification timing and dashboard display order for users. As a technical effect, by using AI to integratively analyze submission timing and event information in a high-dimensional feature space and dynamically optimize the priority of report generation and delivery, the provision unit greatly improves the speed of report generation for important conversations and the overall system processing efficiency compared to conventional uniform report generation order or manual allocation methods, thereby dramatically enhancing user experience and business efficiency. Application fields include creation of important minutes for business meetings, emergency medical records in medical settings, recording of important statements in educational settings, and prioritized conversation recording in counseling settings.

The provision unit can analyze the report by referring to related market data of the conversation when providing the report. For example, the provision unit refers to market data related to the content of the conversation and reflects it in the report. The provision unit may also analyze market trends related to the topic of the conversation and include them in the report. Furthermore, the provision unit may optimize the report based on related market data while considering the flow of the conversation. By analyzing the report by referring to related market data of the conversation, more accurate reports can be provided. Some or all of the above-described processing in the provision unit may be performed using AI, or may be performed without using AI. For example, the provision unit can input related market data into AI and have the AI analyze the report. Specifically, the provision unit integrates input data such as text data of conversation content received from the analysis unit or conversion unit (e.g., “Discussion on market trends”, “Sales analysis of new products”, etc.) and external or internal market databases (e.g., stock price time series data, industry reports, sales statistics, competitor information, trend graphs). Examples of input include “Conversation topic: AI market+market data: Jun. 2024 AI-related stock price trends”, “Conversation topic: new product+market data: year-on-year sales”. The provision unit inputs these data into a large language model with market data reference or a time series analysis model (e.g., LSTM, GRU), and outputs relevance scores between conversation content and market data (e.g., 0.92), market trend summaries, and report optimization parameters (e.g., graph insertion, annotation addition). For example, when “AI market+Jun. 2024 stock price trends” is given as input, the output may be “Market trend: upward trend (score 0.91), graph insertion”. In subsequent processing, the provision unit automatically generates the report reflecting market data and presents it to the user. Furthermore, the provision unit can collect user feedback and market data update history and reflect them in the report optimization algorithm for future reports. As a technical effect, by using AI to integratively analyze conversation content and market data in a high-dimensional feature space and dynamically optimize report content, the provision unit enables highly accurate report generation that reflects the latest market trends and industry information, greatly improving the practicality and business value of the overall conversation quality improvement system compared to conventional manual data reference or static report methods. Application fields include creation of market analysis reports for business meetings, presentation of competitor information in sales activities, decision support in management meetings, and support for economic learning in educational settings.

The provision unit (paid version) can estimate the user's emotion and determine the priority of records to be saved based on the estimated emotion of the user. For example, when the user is nervous, the provision unit (paid version) preferentially saves important conversations. The provision unit (paid version) may also save all conversations equally when the user is relaxed. Furthermore, when the user is in a hurry, the provision unit (paid version) may preferentially save important information in a short time. By determining the priority of records to be saved based on the user's emotion, important conversations can be preferentially saved. Emotion estimation is realized, for example, by using an emotion engine or generative AI with emotion estimation functions. Generative AI may be, for example, a text generation AI (such as LLM) or a multimodal generative AI, but is not limited to these examples. Some or all of the above-described processing in the provision unit (paid version) may be performed using AI, or may be performed without using AI. For example, the provision unit (paid version) can input the user's emotion data into AI and have the AI determine the priority of records to be saved. Specifically, the provision unit (paid version) inputs multimodal data such as the user's voice waveform data, biometric sensor data, and facial images received from the recording unit or analysis unit into an emotion estimation model, and obtains emotion labels (e.g., nervous, relaxed, in a hurry) and emotion intensity scores (e.g., nervousness 0.82). Examples of input include “voice waveform+nervousness 0.82” and “voice waveform+relaxation 0.91”. The provision unit (paid version) combines these emotion estimation results with conversation record data to be saved (e.g., audio files, character string conversation logs, importance scores for each statement, risk scores), and inputs them into a save priority determination algorithm (e.g., multilayer perceptron or decision tree-based classifier). The provision unit (paid version) preferentially saves important conversations when nervousness is high, saves all conversations equally when relaxation is high, and preferentially saves important information in a short time when in a hurry. Outputs include record lists with save priority, priority labels (e.g., priority, normal, deferred), and save action scores (e.g., 0.95). For example, when “nervous (0.82)+conversation importance 0.92” is given as input, the output may be “priority save (0.97)”. In subsequent processing, the provision unit (paid version) automatically organizes records and manages the upper limit of the number of saved records according to save priority, and updates the saved record list on the user interface. As a technical effect, by using AI to integratively evaluate the user's emotional state and the importance of conversation content in a high-dimensional feature space and dynamically optimize save priority, the provision unit (paid version) fundamentally improves computer technology by reducing the user's psychological burden and enabling high-quality saving of important conversations without omission compared to conventional uniform saving methods or manual selection methods. Application fields include recording of important conversations in counseling and coaching settings, patient interaction records in medical and welfare fields, interview records in educational settings, and minutes creation in business meetings.

The provision unit (paid version) can optimize the saving algorithm by referring to past saved data when saving records. For example, the provision unit (paid version) refers to the user's past saved data and reflects it in the current saving. The provision unit (paid version) may also analyze past saved data to improve the accuracy of the current saving algorithm. Furthermore, the provision unit (paid version) may propose the optimal saving method based on the user's past saved data. By optimizing the saving algorithm by referring to past saved data, more accurate saving becomes possible. Some or all of the above-described processing in the provision unit (paid version) may be performed using AI, or may be performed without using AI. For example, the provision unit (paid version) can input past saved data into AI and have the AI optimize the saving algorithm. Specifically, the provision unit (paid version) obtains past saved data for each user (e.g., saved audio files, conversation logs, save action history, save priority scores, archive history when the upper limit of saved records is exceeded, user feedback) from a database and inputs them into a saving algorithm optimization model (e.g., multilayer perceptron with history analysis or saving method recommendation algorithm). Examples of input include “Jun. 1, 2024: priority save of important conversations”, “Jun. 8, 2024: equal save of all records”, “Jun. 15, 2024: archive executed”. The provision unit (paid version) analyzes saving method trends (e.g., characteristics of conversations prioritized for saving in the past, archive frequency), extracts user saving behavior patterns, and optimizes the saving algorithm (e.g., priority-focused type, equal save type, archive-focused type). Outputs include optimized saving algorithm IDs, saving method parameters, and recommended save action lists. For example, when “important conversations have been prioritized for saving in the past three saves” is given as input, the output may be “priority save algorithm”. In subsequent processing, the provision unit (paid version) automatically applies the optimized saving algorithm to improve the efficiency and quality of record saving. Furthermore, the provision unit (paid version) continuously collects new saving behaviors and feedback from users and reflects them in the optimization of the saving algorithm for future saves. As a technical effect, by using AI to integratively analyze past saved data and user behavior history in a high-dimensional feature space and dynamically optimize the saving algorithm, the provision unit (paid version) enables highly accurate record saving and continuous quality improvement tailored to each user's needs and usage trends, greatly improving the usefulness and user satisfaction of the overall conversation quality improvement system compared to conventional static saving methods or manual customization methods. Application fields include long-term saving of 1-on-1 meeting records in companies, management of conversation history for each student in educational settings, record saving for each patient in medical and welfare fields, and quality management in customer support.

The provision unit (paid version) can estimate the user's emotion and adjust the display method of records to be saved based on the estimated emotion of the user. For example, when the user is nervous, the provision unit (paid version) provides a simple and highly visible display method. The provision unit (paid version) may also provide a display method containing detailed information when the user is relaxed. Furthermore, when the user is in a hurry, the provision unit (paid version) may provide a display method that emphasizes key points. By adjusting the display method of records to be saved based on the user's emotion, more appropriate display becomes possible. Emotion estimation is realized, for example, by using an emotion engine or generative AI with emotion estimation functions. Generative AI may be, for example, a text generation AI (such as LLM) or a multimodal generative AI, but is not limited to these examples. Some or all of the above-described processing in the provision unit (paid version) may be performed using AI, or may be performed without using AI. For example, the provision unit (paid version) can input the user's emotion data into AI and have the AI adjust the display method. Specifically, the provision unit (paid version) inputs multimodal data such as the user's voice waveform data, biometric sensor data, and facial images received from the recording unit or analysis unit into an emotion estimation model, and obtains emotion labels (e.g., nervous, relaxed, in a hurry) and emotion intensity scores (e.g., nervousness 0.82). Examples of input include “voice waveform+nervousness 0.82” and “voice waveform+relaxation 0.91”. The provision unit (paid version) combines these emotion estimation results with saved record data (e.g., conversation logs, importance scores for each statement, save date and time, metadata), and inputs them into a display method determination algorithm (e.g., large language model with template selection or display element recommendation algorithm). The provision unit (paid version) automatically selects a simple list display, large font, and design with fewer colors when nervousness is high, generates a rich display with detailed graphs, annotations, and past comparison charts when relaxation is high, and generates a summary display emphasizing only key points when in a hurry. Outputs include display template IDs, display element lists, and display format fit scores (e.g., 0.95). For example, when “nervous (0.82)+saved record list” is given as input, the output may be “simple display (key points only, large font)” or “detailed display (graph+annotation)”. In subsequent processing, the provision unit (paid version) presents the saved records on the user interface in the output display format and collects user browsing history and feedback to reflect in the optimization of display methods for future records. As a technical effect, by using AI to integratively analyze the user's emotional state and saved record content in a high-dimensional feature space and dynamically optimize the display method, the provision unit (paid version) enables flexible and highly efficient information presentation tailored to the user experience and situation, greatly improving the practicality and user satisfaction of the overall conversation quality improvement system compared to conventional uniform display methods or manual switching methods. Application fields include presentation of important records in counseling and coaching settings, patient interaction records in medical and welfare fields, interview records in educational settings, and minutes management in business meetings.

The provision unit (paid version) can weight saved data based on the submission timing of the conversation when saving records. For example, the provision unit (paid version) preferentially saves recent conversations. The provision unit (paid version) may also save past conversations with normal weighting. Furthermore, the provision unit (paid version) may preferentially save conversations related to specific events immediately after the event. By weighting saved data based on the submission timing of the conversation, important conversations can be preferentially saved. Some or all of the above-described processing in the provision unit (paid version) may be performed using AI, or may be performed without using AI. For example, the provision unit (paid version) can input submission timing data into AI and have the AI perform weighting of saved data. Specifically, the provision unit (paid version) receives input data such as submission time data for conversation records (e.g., timestamp, event ID, conversation type, conversation content features). Examples of input include “Jun. 1, 2024, 10:00, Conversation A”, “Jun. 2, 2024, 15:30, Conversation B (event ID=E 123)”. The provision unit (paid version) inputs these data into a time series weighting determination model (e.g., recurrent neural network or decision tree-based classifier), and automatically determines weighting scores for saved data (e.g., 0.95, 0.75, 0.45) and save priority labels (e.g., priority, normal, deferred) based on submission timing and event information. Outputs include saved record lists with weighting, priority labels, and weighting scores. For example, when “Conversation A (latest)”, “Conversation B (immediately after event)” are given as input, the output may be “Conversation A: weight 0.95”, “Conversation B: weight 0.92”. In subsequent processing, the provision unit (paid version) automatically organizes saved records and manages the upper limit of the number of saved records according to weighting scores, and reorders the saved record list on the user interface. As a technical effect, by using AI to integratively analyze submission timing and event information in a high-dimensional feature space and dynamically optimize the weighting of saved data, the provision unit (paid version) greatly improves the prioritization of saving important conversations and the overall system storage efficiency compared to conventional uniform saving methods or manual allocation methods, thereby dramatically enhancing user experience and business efficiency. Application fields include saving important minutes for business meetings, emergency medical records in medical settings, recording of important statements in educational settings, and prioritized conversation recording in counseling settings.

The provision unit (growth trend confirmation) can estimate the user's emotion and adjust the display method of the growth trend based on the estimated emotion of the user. For example, when the user is nervous, the provision unit (growth trend confirmation) displays a simple and highly visible growth trend. The provision unit (growth trend confirmation) may also display a growth trend containing detailed information when the user is relaxed. Furthermore, when the user is in a hurry, the provision unit (growth trend confirmation) may display a growth trend that emphasizes key points. By adjusting the display method of the growth trend based on the user's emotion, more appropriate display of the growth trend becomes possible. Emotion estimation is realized, for example, by using an emotion engine or generative AI with emotion estimation functions. Generative AI may be, for example, a text generation AI (such as LLM) or a multimodal generative AI, but is not limited to these examples. Some or all of the above-described processing in the provision unit (growth trend confirmation) may be performed using AI, or may be performed without using AI. For example, the provision unit (growth trend confirmation) can input the user's emotion data into AI and have the AI adjust the display method. Specifically, the provision unit (growth trend confirmation) inputs multimodal data such as the user's voice waveform data, biometric sensor data, and facial images received from the recording unit or analysis unit into an emotion estimation model, and obtains emotion labels (e.g., nervous, relaxed, in a hurry) and emotion intensity scores (e.g., nervousness 0.82). Examples of input include “voice waveform+nervousness 0.82” and “voice waveform+relaxation 0.91”. The provision unit (growth trend confirmation) combines these emotion estimation results with growth trend data (e.g., time series score arrays, growth point lists, past comparison graphs), and inputs them into a display method determination algorithm (e.g., large language model with template selection or display element recommendation algorithm). The provision unit (growth trend confirmation) automatically selects a simple graph or key point list when nervousness is high, generates a rich display with detailed graphs, annotations, and past comparison charts when relaxation is high, and generates a summary display emphasizing only key points when in a hurry. Outputs include display template IDs, display element lists, and display format fit scores (e.g., 0.95). For example, when “nervous (0.82)+growth trend data” is given as input, the output may be “simple display (key points only, large font)” or “detailed display (graph+annotation)”. In subsequent processing, the provision unit (growth trend confirmation) presents the growth trend on the user interface in the output display format and collects user browsing history and feedback to reflect in the optimization of display methods for future growth trend displays. As a technical effect, by using AI to integratively analyze the user's emotional state and growth trend data in a high-dimensional feature space and dynamically optimize the display method, the provision unit (growth trend confirmation) enables flexible and highly efficient information presentation tailored to the user experience and situation, greatly improving the practicality and user satisfaction of the overall conversation quality improvement system compared to conventional uniform display methods or manual switching methods. Application fields include presentation of growth trends in counseling and coaching settings, patient care records in medical and welfare fields, student guidance records in educational settings, and human resource development records in business meetings.

The provision unit (growth trend confirmation) can optimize the current growth by referring to past growth data when confirming the growth trend. For example, the provision unit (growth trend confirmation) refers to the user's past growth data and reflects it in the current growth. The provision unit (growth trend confirmation) may also analyze past growth data to improve the accuracy of the current growth. Furthermore, the provision unit (growth trend confirmation) may propose the optimal growth method based on the user's past growth data. By optimizing the current growth by referring to past growth data, more accurate confirmation of the growth trend becomes possible. Some or all of the above-described processing in the provision unit (growth trend confirmation) may be performed using AI, or may be performed without using AI. For example, the provision unit (growth trend confirmation) can input past growth data into AI and have the AI optimize the growth. Specifically, the provision unit (growth trend confirmation) obtains past growth data for each user (e.g., time series score arrays, growth point lists, past comparison graphs, feedback history, growth method selection history) from a database and inputs them into a growth trend optimization model (e.g., large language model with history analysis or growth method recommendation algorithm). Examples of input include “Jun. 1, 2024: risk score 0.85”, “Jun. 8, 2024: risk score 0.65”, “Jun. 15, 2024: risk score 0.42”, “trend of aggressive statement ratio in the last 10 1-on-1 meetings”. The provision unit (growth trend confirmation) analyzes growth trends (e.g., growth slope value, growth degree score), extracts growth points (e.g., “Since May, negative statements have greatly decreased”), and proposes optimal growth methods (e.g., feedback enhancement type, self-evaluation emphasis type). Outputs include optimized growth trend graphs, recommended growth method lists, and growth degree scores (e.g., +0.35). For example, when “risk score is decreasing in the past three growth data” is given as input, the output may be “growth score: +0.35, recommended method: feedback enhancement type”. In subsequent processing, the provision unit (growth trend confirmation) presents the optimized growth trend display and growth method proposals to the user, and continuously collects new growth actions and feedback from users to reflect in the optimization of growth trends for future confirmations. As a technical effect, by using AI to integratively analyze past growth data and user behavior history in a high-dimensional feature space and dynamically optimize growth trends and growth methods, the provision unit (growth trend confirmation) enables highly accurate confirmation of growth trends and continuous growth support tailored to each user's needs and usage trends, greatly improving the usefulness and user satisfaction of the overall conversation quality improvement system compared to conventional static growth displays or manual customization methods. Application fields include human resource development in companies, student guidance in educational settings, patient care in medical and welfare fields, and self-development support services.

The provision unit (growth trend confirmation) can estimate the user's emotion and determine the priority of the growth trend based on the estimated emotion of the user. For example, when the user is nervous, the provision unit (growth trend confirmation) emphasizes and displays important growth points. When the user is relaxed, the provision unit (growth trend confirmation) can display overall growth evenly. Furthermore, when the user is in a hurry, the provision unit (growth trend confirmation) can display a growth trend that concisely summarizes the key points. By determining the priority of the growth trend based on the user's emotion, important growth points can be emphasized and displayed. Emotion estimation is realized, for example, by using an emotion estimation function such as an emotion engine or generative AI. Generative AI may include, for example, text generation AI (such as LLM) or multimodal generative AI, but is not limited to these examples. Some or all of the above-described processing in the provision unit (growth trend confirmation) may be performed using AI or may be performed without using AI. For example, the provision unit (growth trend confirmation) can input the user's emotion data to AI and have the AI execute the priority determination. Specifically, the provision unit (growth trend confirmation) inputs multimodal data such as the user's voice waveform data, biometric sensor data, and facial images received from the recording unit or analysis unit into an emotion estimation model to obtain emotion labels (e.g., nervous, relaxed, in a hurry) and emotion intensity scores (e.g., nervousness 0.82). Examples of input include “voice waveform+nervousness 0.82” and “voice waveform+relaxation 0.91”. The provision unit (growth trend confirmation) combines these emotion estimation results with growth trend data (e.g., growth point list, time-series score array, importance score) and inputs them into a priority determination algorithm (e.g., multilayer perceptron or decision tree-based classifier). When the nervousness is high, the provision unit (growth trend confirmation) emphasizes important growth points; when the relaxation is high, it displays overall growth evenly; and when the user is in a hurry, it displays a growth trend that concisely summarizes only the key points. Outputs include a prioritized growth trend list, emphasis score (e.g., 0.95), and display labels (e.g., important, normal, key points). For example, when “nervousness (0.82)+growth point list” is input, an output such as “important point emphasis type growth trend” can be obtained. As a subsequent process, the provision unit (growth trend confirmation) can display the prioritized growth trend in the optimal format on the user interface and collect the user's browsing history and feedback to reflect in the priority determination algorithm for future use. As a technical effect, by using AI to integratively analyze the user's emotional state and growth trend data in a high-dimensional feature space and dynamically optimize the priority of the growth trend, the provision unit (growth trend confirmation) enables flexible and highly efficient information presentation tailored to the user experience and situation, greatly improving the practicality and user satisfaction of the overall conversation quality improvement system compared to conventional uniform display methods or manual editing methods. Application fields include growth trend presentation in counseling and coaching settings, patient care records in medical and welfare fields, student guidance records in educational settings, and human resource development records in business meetings.

The provision unit (growth trend confirmation) can weight growth data based on the submission timing of the conversation when confirming the growth trend. For example, the provision unit (growth trend confirmation) prioritizes the display of recent growth data. Past growth data can also be displayed with normal weighting. Furthermore, growth data related to specific events can be prioritized for display immediately after the event. By weighting growth data based on the submission timing of the conversation, important growth data can be prioritized for display. Some or all of the above-described processing in the provision unit (growth trend confirmation) may be performed using AI or may be performed without using AI. For example, the provision unit (growth trend confirmation) can input submission timing data to AI and have the AI execute the weighting of growth data. Specifically, the provision unit (growth trend confirmation) receives submission time data for growth data (e.g., timestamp, event ID, growth type, growth content feature) as input data. Examples of input include “Jun. 1, 2024, 10:00 Growth Data A” and “Jun. 2, 2024, 15:30 Growth Data B (event ID=E123)”. The provision unit (growth trend confirmation) inputs these data into a time-series weighting determination model (e.g., recurrent neural network or decision tree-based classifier) and automatically determines weighting scores for growth data (e.g., 0.95, 0.75, 0.45) and display priority labels (e.g., priority, normal, deferred) based on submission timing and event information. Outputs include a weighted growth data list, priority labels, and weighting scores. For example, when “Growth Data A (latest)” and “Growth Data B (immediately after event)” are input, outputs such as “Growth Data A: weight 0.95” and “Growth Data B: weight 0.92” can be obtained. As a subsequent process, the provision unit (growth trend confirmation) automatically organizes growth data and optimizes display order according to weighting scores, and sorts the growth trend list on the user interface. As a technical effect, by using AI to integratively analyze submission timing and event information of growth data in a high-dimensional feature space and dynamically optimize the weighting of growth data, the provision unit (growth trend confirmation) enables prioritized display of important growth data and greatly improves the efficiency of information presentation for the entire system, as well as user experience and operational efficiency, compared to conventional uniform display methods or manual assignment methods. Application fields include human resource development records in business meetings, patient care records in medical settings, student guidance records in educational settings, and growth trend presentation in counseling settings.

The system according to the embodiment is not limited to the above examples and can be variously modified as follows. Specifically, the system can flexibly change and expand the types of AI models, algorithms, data flows, input/output specifications, and hardware configurations for each component: recording unit, conversion unit, analysis unit, and provision unit. For example, the recording unit can target multimodal data such as images, videos, biometric signals, location information, and sensor data in addition to voice; the conversion unit can implement various conversion processes such as image recognition, natural language generation, summarization, and translation in addition to speech recognition. The analysis unit can apply various analysis algorithms such as emotion analysis, utterance intent estimation, anomaly detection, topic classification, and attribute estimation in addition to psychological safety evaluation; the provision unit can realize various output methods such as dashboard UI, real-time feedback, API integration, and data linkage with external systems in addition to report generation. Regarding AI models, various architectures such as convolutional neural networks, recurrent neural networks, Transformers, graph neural networks, self-supervised learning models, and reinforcement learning models can be selected, and learning methods and parameter optimization techniques can be flexibly changed according to tasks and data characteristics. Furthermore, hardware configurations such as cloud distributed processing, edge AI, GPU clusters, and FPGA accelerators can be selected and optimized according to system scalability, real-time requirements, and security requirements. With these diverse modifications and expansions, the system enables flexible and highly efficient information processing, analysis, and feedback tailored to the usage environment, business requirements, user attributes, and data characteristics, achieving essential improvements in computer technology and broad applicability compared to conventional conversation quality improvement systems with single-function and fixed configurations. Application fields include business meetings, educational settings, medical and welfare fields, customer support, self-development support, on-site work records, and research and development support.

The recording unit can monitor the user's health status when recording the user's voice and adjust the recording method according to the health status. For example, when the user is tired, the recording unit can limit the voice recording to a short duration. When the user is healthy, the normal recording method can be applied. Furthermore, when the user is ill, the recording unit can temporarily suspend voice recording and wait for the user's recovery. By adjusting the voice recording method according to the user's health status, the user's burden can be reduced. Specifically, the recording unit obtains real-time health indicator data from wearable devices or biometric sensors (e.g., heart rate, blood oxygen saturation, skin temperature, activity tracker) to monitor the user's health status. Examples of input include “heart rate 110 bpm+decreased activity” and “blood oxygen 95%+body temperature 37.8° C.”. The recording unit inputs these health indicator data into a health status estimation model (e.g., multilayer perceptron or time-series analysis model) and generates output such as health status labels (e.g., healthy, fatigued, ill) and health risk scores (e.g., fatigue 0.82, health 0.95). Examples of output include “fatigue (0.82)”, “health (0.95)”, and “illness (0.91)”. Based on these outputs, the recording unit performs automatic control of recording time (e.g., limit to within 10 minutes when fatigued), temporary suspension of recording (e.g., stop recording when ill), and optimization of recording resumption timing (e.g., resume when health score is 0.9 or higher). Furthermore, the recording unit can collect the user's health status history and feedback and reflect them in the recording method optimization algorithm. As a technical effect, by using AI to accurately estimate the user's health status and dynamically optimize the recording method, the recording unit greatly reduces the user's physical and psychological burden and improves long-term recording quality and user experience compared to conventional uniform recording methods or manual adjustment methods. Application fields include patient records in medical and welfare fields, student health management in educational settings, employee care in business settings, and self-management support services.

The conversion unit can adjust the conversion algorithm by considering the user's speech rate when converting recorded voice into a character string. For example, when the user speaks quickly, the conversion unit can apply a fast conversion algorithm. When the user speaks slowly, the conversion unit can apply a conversion algorithm that emphasizes accuracy. Furthermore, when the user's speech rate fluctuates, the conversion unit can adjust the algorithm in real time. By adjusting the conversion algorithm according to the user's speech rate, more accurate character strings can be generated. Specifically, the conversion unit receives voice tensors from the recording unit (e.g., waveform data of 16,000 samples per second or spectrograms of 128 dimensions ×100 frames) as input and estimates frame-by-frame speech rate indicators (e.g., phoneme interval, speech rate score) using an acoustic feature extraction model (e.g., convolutional neural network). Examples of input include “voice waveform+speech rate: 7 phonemes per second” and “voice waveform+speech rate: 3 phonemes per second”. Based on the speech rate estimation results, the conversion unit dynamically adjusts parameters such as decoder beam width, search depth, and noise suppression in the speech recognition model (e.g., Transformer-based speech recognition model or RNN). For fast speech, the beam width is narrowed to prioritize speed; for slow speech, the beam width is widened to prioritize accuracy. Outputs include UTF-8 encoded character strings (e.g., “Thank you for today”), conversion confidence scores (e.g., 0.98), and conversion speed (e.g., 0.3 seconds per sentence). For example, when “voice waveform+speech rate: 7 phonemes per second” is input, an output such as “fast conversion: Thank you for today (0.95)” can be obtained. As a subsequent process, the conversion unit passes the output character string and confidence score to the analysis unit for psychological safety evaluation and statement trend analysis. As a technical effect, by using AI to dynamically optimize the parameters of the speech recognition model according to the user's speech rate, the conversion unit enables high-precision and high-efficiency character string generation tailored to the user experience and situation, greatly improving the reliability and practicality of the overall conversation quality improvement system compared to conventional uniform conversion accuracy settings or manual adjustment methods. Application fields include minutes creation for business meetings, lesson records in educational settings, medical records in medical settings, and conversation records in counseling settings.

The analysis unit can adjust the focus of analysis based on the topic of the conversation when analyzing the converted character string. For example, in business conversations, the analysis unit can focus on professional language usage. In casual conversations, the analysis unit can focus on relaxed language usage. Furthermore, in academic conversations, the analysis unit can focus on the use of technical terms. By adjusting the focus of analysis according to the topic of the conversation, more appropriate analysis results can be provided. Specifically, the analysis unit integrates character string data received from the conversion unit (e.g., “Today we will discuss the progress of the meeting”, “Discussion on the latest trends in AI technology”) and conversation topic labels (e.g., business, casual, academic, medical, education) as input data. Examples of input include “Topic: business+statement: ‘Project progress report’”, “Topic: casual+statement: ‘How have you been lately?’”, “Topic: academic+statement: ‘Application of neural networks’”. The analysis unit inputs these data into a topic-conditioned large language model or topic-weighted classifier (e.g., multilayer perceptron, decision tree) and applies topic-weighted analysis criteria (e.g., professionalism, relaxation, technical term frequency) to evaluate and classify statements. Outputs include labels for each statement (e.g., 0=professional, 1=casual, 2=academic), focus scores (e.g., 0.92), and analysis result lists. For example, when “Topic: business+statement: ‘Project progress report’” is input, an output such as “High professionalism (0.95)” can be obtained. As a subsequent process, the analysis unit passes the output analysis results to the provision unit for report generation and feedback presentation. As a technical effect, by using AI to integratively analyze conversation topics and statement content in a high-dimensional feature space and dynamically optimize the focus of analysis, the analysis unit enables high-precision analysis results tailored to the usage scene and purpose, greatly improving the practicality and user satisfaction of the overall conversation quality improvement system compared to conventional uniform analysis criteria or manual switching methods. Application fields include minutes analysis for business meetings, lesson records in educational settings, presentation records for academic conferences, medical records in medical settings, and support for casual communication.

The provision unit can adjust the format of the report according to the user's learning style when providing the analysis result as a report. For example, for visual learners, the provision unit can provide reports that use many graphs and diagrams. For auditory learners, the provision unit can provide reports that include explanations in audio. Furthermore, for experiential learners, the provision unit can provide reports that include interactive elements. By adjusting the format of the report according to the user's learning style, more effective learning can be supported. Specifically, the provision unit integrates the user's learning style information (e.g., visual, auditory, experiential, questionnaire results, past browsing history) and report generation data received from the analysis unit (e.g., statement labels, risk scores, importance scores, time-series data) as input data. Examples of input include “Learning style: visual+report data”, “Learning style: auditory+report data”, “Learning style: experiential+report data”. The provision unit inputs these data into a learning style adaptive report generation model (e.g., template selection large language model or multimodal output engine) and automatically selects the optimal report format for each learning style (e.g., graph-focused, audio explanation, interactive UI). Outputs include report template ID, output format (e.g., PDF, HTML, audio file, interactive dashboard), and display element list. For example, when “Learning style: visual+report data” is input, an output such as “graph-focused report” can be obtained. As a subsequent process, the provision unit automatically generates and presents reports optimized for the user's learning style. Furthermore, the provision unit can collect user feedback and browsing history and reflect them in the report format optimization algorithm for future use. As a technical effect, by using AI to integratively analyze the user's learning style and report content in a high-dimensional feature space and dynamically optimize the report format, the provision unit can maximize learning effectiveness and satisfaction for each user compared to conventional uniform report formats or manual switching methods. Application fields include individualized instruction support in educational settings, corporate human resource development, patient education in medical and welfare fields, and self-development support services.

The provision unit can collect user feedback when providing a report and reflect it in the next report. For example, if the user prefers a specific report format, the provision unit can prioritize providing that format. If the user points out improvements to the report content, the provision unit can reflect those improvements in the next report. Furthermore, if the user wants to adjust the frequency of reports, the provision unit can change the report frequency according to the user's request. By reflecting user feedback, more suitable reports can be provided to the user. Specifically, the provision unit collects user feedback data after report viewing (e.g., report format selection, content evaluation score, improvement request text, desired viewing frequency) and inputs them into a feedback analysis model (e.g., natural language processing model or history analysis classifier). Examples of input include “Format: graph type is easy to see”, “Content: please emphasize key points more”, “Frequency: prefer once a week”. Based on these feedback data, the provision unit automatically optimizes template selection, content emphasis parameters, and provision frequency settings for the next report generation. Outputs include next report template ID, content emphasis score, and provision frequency setting value. For example, when “graph type is easy to see+request for key point emphasis” is input, an output such as “graph-focused type+key point emphasis score 0.9” can be obtained. As a subsequent process, the provision unit automatically generates and presents the optimized report to the user. Furthermore, by continuously collecting new user feedback and reflecting it in the report generation algorithm, continuous quality improvement and maximization of user satisfaction can be achieved. As a technical effect, by using AI to integratively analyze user feedback and report content in a high-dimensional feature space and dynamically optimize report content, format, and frequency, the provision unit enables high-precision report generation and continuous quality improvement tailored to each user's needs and usage trends, greatly improving the usefulness and user satisfaction of the overall conversation quality improvement system compared to conventional static report methods or manual customization methods. Application fields include support for 1-on-1 interviews in companies, individualized instruction records in educational settings, patient progress reports in medical and welfare fields, and quality management in customer support.

The recording unit can estimate the user's emotion and adjust the voice recording method based on the estimated emotion of the user. For example, when the user is excited, the recording unit can record voice quickly. When the user is calm, the recording unit can record voice at a normal speed. Furthermore, when the user is sad, the recording unit can temporarily suspend voice recording and wait until the user's emotion stabilizes. By adjusting the voice recording method based on the user's emotion, voice can be recorded at more appropriate timing. Specifically, the recording unit inputs multimodal data such as the user's voice waveform data (e.g., 16,000 samples of PCM data per second), biometric sensor data (e.g., heart rate, skin conductance), and facial images (e.g., facial feature points extracted from camera images) into an emotion estimation model (e.g., Transformer-based neural network integrating voice, image, and biometric signals) and generates output such as emotion labels (e.g., excitement, calmness, sadness) and emotion intensity scores (e.g., excitement 0.82, calmness 0.91, sadness 0.75). Examples of input include “voice waveform+heart rate 110 bpm+facial expression: smile”, “voice waveform+heart rate 60 bpm+facial expression: neutral”, “voice waveform+heart rate 70 bpm+facial expression: tears”. Based on these outputs, the recording unit performs automatic control of recording start timing (e.g., immediate recording when excited, normal recording when calm, temporary suspension when sad), dynamic adjustment of recording time, and optimization of notification timing for recording start to the user. As a technical effect, by using AI to accurately estimate the user's emotional state and dynamically optimize the recording method, the recording unit reduces the user's psychological burden and enables more natural and high-quality voice data acquisition compared to conventional uniform recording start methods or manual adjustment methods. Application fields include conversation recording in counseling and coaching settings, patient response records in medical and welfare fields, and interview records in educational settings.

The conversion unit can estimate the user's emotion and adjust the character string conversion method based on the estimated emotion of the user. For example, when the user is angry, the conversion unit can convert to a character string with suppressed emotion. When the user is happy, the conversion unit can convert to a character string with emphasized emotion. Furthermore, when the user is sad, the conversion unit can convert to a character string that reflects the emotion. By adjusting the character string conversion method based on the user's emotion, more appropriate character strings can be generated. Specifically, the conversion unit integrates voice tensors received from the recording unit (e.g., waveform data of 16,000 samples per second or spectrograms of 128 dimensions×100 frames) and emotion estimation results (e.g., anger 0.85, joy 0.91, sadness 0.78) as input data. Examples of input include “voice waveform+anger 0.85”, “voice waveform+joy 0.91”, “voice waveform+sadness 0.78”. The conversion unit inputs these data into a Transformer-based speech recognition model or large language model and dynamically adjusts decoder emotion suppression/emphasis parameters and generation temperature according to the emotional state. For example, when anger is strong, emotion suppression tokens are inserted to generate sentences with suppressed emotional expression; when joy is strong, emotion emphasis tokens are inserted to generate sentences with emphasized emotional expression; and when sadness is strong, emotion reflection tokens are inserted to generate sentences that reflect the emotion. Outputs include UTF-8 encoded character strings (e.g., “Calm expression: Thank you for today”, “Emotion emphasized: I am very happy today”, “Emotion reflected: I am a little sad today”), and emotion reflection score (e.g., 0.88). As a subsequent process, the conversion unit can automatically optimize and personalize emotion control parameters based on user feedback and usage history. As a technical effect, by using AI to integratively analyze the user's emotional state and voice content in a high-dimensional feature space and dynamically optimize the emotion reflection degree of character string generation, the conversion unit enables flexible and high-precision character string generation tailored to the user experience and situation, greatly improving the practicality and user satisfaction of the overall conversation quality improvement system compared to conventional uniform output methods or manual editing methods. Application fields include minutes creation for business meetings, lesson records in educational settings, medical records in medical settings, and emotion-reflecting records in counseling settings.

The analysis unit can estimate the user's emotion and adjust the analysis accuracy based on the estimated emotion of the user. For example, when the user is nervous, the analysis unit can perform analysis with strict criteria. When the user is relaxed, the analysis unit can perform analysis with normal criteria. Furthermore, when the user is in a hurry, the analysis unit can relax the criteria to perform rapid analysis. By adjusting the analysis accuracy based on the user's emotion, more appropriate analysis results can be provided. Specifically, the analysis unit inputs multimodal data such as the user's voice waveform data, biometric sensor data, and facial images received from the recording unit or conversion unit into an emotion estimation model to obtain emotion labels (e.g., nervous, relaxed, in a hurry) and emotion intensity scores (e.g., nervousness 0.82). Examples of input include “voice waveform+nervousness 0.82” and “voice waveform+relaxation 0.91”. The analysis unit combines these emotion estimation results with character string data received from the conversion unit (e.g., “You are useless”, “Thank you for today”) and inputs them into an analysis accuracy adjustment algorithm (e.g., rule-based judgment with threshold control, large language model with emotion-dependent weighting). When nervousness is high, the analysis unit tightens the threshold for psychological safety risk; when relaxation is high, it applies normal thresholds; and when the user is in a hurry, it narrows down analysis items to speed up processing, dynamically adjusting analysis accuracy. Outputs include labels for each statement (e.g., 0=safe, 1=aggressive, 2=negative), risk scores (e.g., 0.85), and analysis accuracy labels (e.g., strict, normal, relaxed). For example, when “nervousness (0.82)+statement: ‘Can't you even do that?’” is input, an output such as “Aggressive (score 0.92, accuracy: strict)” can be obtained. As a subsequent process, the analysis unit generates feedback for each statement, creates reports, and displays heatmaps based on the output labels and scores. As a technical effect, by using AI to dynamically optimize analysis accuracy in a high-dimensional feature space according to the user's emotional state, the analysis unit enables flexible and high-precision statement evaluation tailored to the user experience and situation, greatly improving the reliability and usefulness of the overall conversation quality improvement system compared to conventional uniform analysis criteria or manual adjustment methods. Application fields include conversation analysis in counseling and coaching settings, patient response records in medical and welfare fields, interview records in educational settings, and minutes analysis for business meetings.

The provision unit can estimate the user's emotion and adjust the content of the report based on the estimated emotion of the user. For example, when the user is nervous, the provision unit can provide a report that emphasizes important points. When the user is relaxed, the provision unit can provide overall information evenly. Furthermore, when the user is in a hurry, the provision unit can provide a report that concisely summarizes the key points. By adjusting the content of the report based on the user's emotion, more appropriate reports can be provided. Specifically, the provision unit inputs multimodal data such as the user's voice waveform data, biometric sensor data, and facial images received from the recording unit or analysis unit into an emotion estimation model to obtain emotion labels (e.g., nervous, relaxed, in a hurry) and emotion intensity scores (e.g., nervousness 0.82). Examples of input include “voice waveform+nervousness 0.82” and “voice waveform+relaxation 0.91”. The provision unit combines these emotion estimation results with report generation data received from the analysis unit (e.g., importance score for each statement, risk score, key point list) and inputs them into a content adjustment algorithm (e.g., weighted template engine or large language model with content emphasis). When nervousness is high, the provision unit emphasizes important points; when relaxation is high, it arranges overall information evenly; and when the user is in a hurry, it generates a report that concisely summarizes only the key points. Outputs include report files with emphasis elements, content emphasis score (e.g., 0.95), and display labels (e.g., important, normal, key points). For example, when “nervousness (0.82)+key point list” is input, an output such as “key point emphasis type report (important points displayed in red)” can be obtained. As a subsequent process, the provision unit displays the generated report in the optimal format on the user interface and collects the user's browsing history and feedback to reflect in the content adjustment algorithm for future use. As a technical effect, by using AI to integratively analyze the user's emotional state and report content in a high-dimensional feature space and dynamically optimize the report content, the provision unit enables flexible and highly efficient information presentation tailored to the user experience and situation, greatly improving the practicality and user satisfaction of the overall conversation quality improvement system compared to conventional uniform emphasis methods or manual editing methods. Application fields include presentation of important analysis results in counseling and coaching settings, patient response records in medical and welfare fields, interview records in educational settings, and minutes analysis for business meetings.

The provision unit can estimate the user's emotion and adjust the display method of the report based on the estimated emotion of the user. For example, when the user is nervous, the provision unit can provide a simple and highly visible report. When the user is relaxed, the provision unit can provide a report containing detailed information. Furthermore, when the user is in a hurry, the provision unit can provide a report that highlights the key points. By adjusting the display method of the report based on the user's emotion, more appropriate reports can be provided. Specifically, the provision unit inputs multimodal data such as the user's voice waveform data, biometric sensor data, and facial images received from the recording unit or analysis unit into an emotion estimation model to obtain emotion labels (e.g., nervous, relaxed, in a hurry) and emotion intensity scores (e.g., nervousness 0.82). Examples of input include “voice waveform+nervousness 0.82” and “voice waveform+relaxation 0.91”. The provision unit combines these emotion estimation results with report generation data received from the analysis unit (e.g., labels for each statement, risk score, importance score, time-series data) and inputs them into a display method determination algorithm (e.g., multilayer perceptron, decision tree-based classifier, or template selection large language model). When nervousness is high, the provision unit automatically selects a simple layout, large font, and a design with fewer colors; when relaxation is high, it generates a rich report including detailed graphs, annotations, and past comparison charts; and when the user is in a hurry, it generates a summary report that emphasizes only the key points. Outputs include report files such as PDF, HTML, or dashboard UI, display template ID, and display element list (e.g., graphs, text, key point list). For example, when “nervousness (0.82)+analysis result list” is input, outputs such as “simple report (key points only, large font)” or “detailed report (graph+annotation)” can be obtained. As a subsequent process, the provision unit displays the output report in the optimal format on the user interface and collects the user's browsing history and feedback to reflect in the display method optimization for future use. As a technical effect, by using AI to integratively analyze the user's emotional state and report content in a high-dimensional feature space and dynamically optimize the report display method, the provision unit enables flexible and highly efficient information presentation tailored to the user experience and situation, greatly improving the practicality and user satisfaction of the overall conversation quality improvement system compared to conventional uniform report display or manual switching methods. Application fields include presentation of important analysis results in counseling and coaching settings, patient response records in medical and welfare fields, interview records in educational settings, and minutes analysis for business meetings.

Below is a brief explanation of the processing flow of Example of the Embodiment. Specifically, the system implements a series of data flows in which the recording unit records the user's voice in high quality, the conversion unit converts the voice tensor into a character string using convolutional neural networks or Transformer-based speech recognition models, the analysis unit analyzes the converted character string for psychological safety and statement trends using large language models or rule-based engines, and the provision unit automatically generates and presents the analysis results in report formats such as PDF, HTML, or dashboard UI. Each step clearly defines the input/output specifications of AI models (e.g., voice waveform, spectrogram, token ID sequence, label, score), algorithm details (e.g., beam search, CTC decoder, attention mechanism), and subsequent processing (e.g., feedback generation, heatmap display, storage and archive management). By integratively analyzing and optimizing diverse context information such as the user's emotion, conversation content, usage history, health status, and learning style in a high-dimensional feature space, the system achieves dramatic technical effects in accuracy, efficiency, flexibility, and user experience compared to conventional simple automation or manual processing. Application fields include business meetings, educational settings, medical and welfare fields, customer support, self-development support, on-site work records, and research and development support.

Step 1: The recording unit records the user's voice. The user's voice includes conversation voice, instruction voice, and emotional expression voice. The recording unit records the user's voice in high quality for use in subsequent processing. Step 2: The conversion unit converts the voice recorded by the recording unit into a character string. Specific methods include speech recognition technology and specific algorithms. The conversion unit uses speech recognition technology to convert voice into a character string and can also apply specific algorithms to improve conversion accuracy. Step 3: The analysis unit analyzes the character string converted by the conversion unit. Specific methods include text analysis technology and analysis algorithms. The analysis unit uses text analysis technology to analyze the character string and identifies statements that impair psychological safety. Step 4: The provision unit provides the analysis result obtained by the analysis unit as a report. The format and provision method of the report include PDF format, web page format, and email transmission. The provision unit provides the analysis result as a report in PDF format or web page format. Specifically, in Step 1, the recording unit records voice at a high sampling rate such as 16 kHz/24 bit and generates a preprocessed voice tensor with noise removal and normalization. In Step 2, the conversion unit inputs the voice tensor and extracts acoustic features using a CNN or Transformer-based speech recognition model, and generates the optimal character string sequence using beam search or a CTC decoder. In Step 3, the analysis unit tokenizes the character string obtained from the conversion unit and outputs psychological safety risk and statement trend as labels and scores using a large language model such as BERT or Transformer-based models. In Step 4, the provision unit automatically generates and presents reports such as PDF, HTML, or dashboard UI based on the output of the analysis unit. Each step clearly defines examples of AI model input/output (e.g., voice waveform→character string, character string→label/score, label/score→report), algorithm details (e.g., attention mechanism, template engine), and subsequent processing (e.g., feedback generation, storage and archive management). By integratively analyzing and optimizing diverse context information such as the user's emotion, conversation content, and usage history in a high-dimensional feature space, the system achieves dramatic technical effects in accuracy, efficiency, flexibility, and user experience compared to conventional simple automation or manual processing. Application fields include business meetings, educational settings, medical and welfare fields, customer support, self-development support, on-site work records, and research and development support.

The specific processing unit 290 sends the results of specific processing to the smart device 14. In the smart device 14, the control unit 46A causes the output device 40 to output the results of specific processing. The microphone 38B acquires voice indicating user input in response to the results of specific processing. The control unit 46A sends the voice data indicating user input acquired by the microphone 38B to the data processing device 12. In the data processing device 12, the specific processing unit 290 acquires the voice data.

The data generation model 58 is a so-called generative AI (Artificial Intelligence). An example of the data generation model 58 is a generative AI such as ChatGPT (registered trademark) (Internet search <URL:https://openai.com/blog/chatgpt>). The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

Moreover, the processing by the data processing system 10 described above is executed by the specific processing unit 290 of the data processing device 12 or the control unit 46A of the smart device 14, but it may be executed by both the specific processing unit 290 of the data processing device 12 and the control unit 46A of the smart device 14. Additionally, the specific processing unit 290 of the data processing device 12 acquires or collects necessary information for processing from the smart device 14 or external devices, and the smart device 14 acquires or collects necessary information for processing from the data processing device 12 or external devices.

Each of the plurality of elements including the aforementioned recording unit, conversion unit, analysis unit, and provision unit is implemented by at least one of, for example, the smart device 14 and the data processing apparatus 12. For example, the recording unit records the user's voice using the microphone 38B of the smart device 14. The conversion unit converts the voice into a character string by, for example, a specific processing unit 290 of the data processing apparatus 12. The analysis unit analyzes the character string by, for example, the specific processing unit 290 of the data processing apparatus 12 and identifies statements that impair psychological safety. The provision unit provides the analysis result as a report by, for example, a control unit 46A of the smart device 14. The correspondence between each unit and the device or control unit is not limited to the examples described above and various modifications are possible.

### Second Embodiment

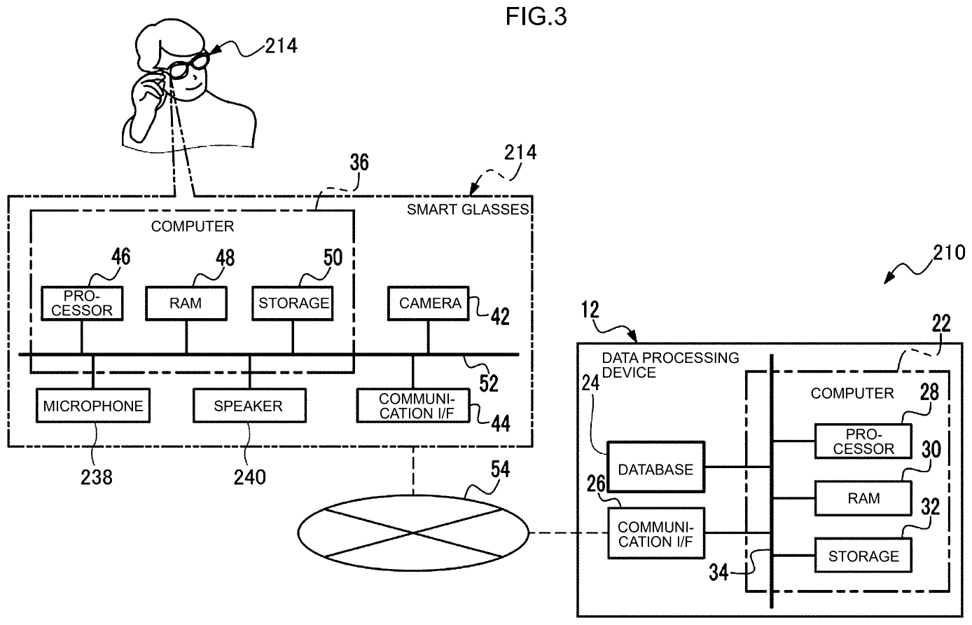

FIG. 3 shows an example configuration of a data processing system 210 according to the second embodiment.

As shown in FIG. 3, the data processing system 210 comprises a data processing device 12 and smart glasses 214. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The smart glasses 214 comprise a computer 36, a microphone 238, a speaker 240, a camera 42, and a communication I/F 44. The computer 36 comprises a processor 46, RAM 48, and storage 50. The processor 46, RAM 48, and storage 50 are connected to a bus 52. The microphone 238, speaker 240, and camera 42 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

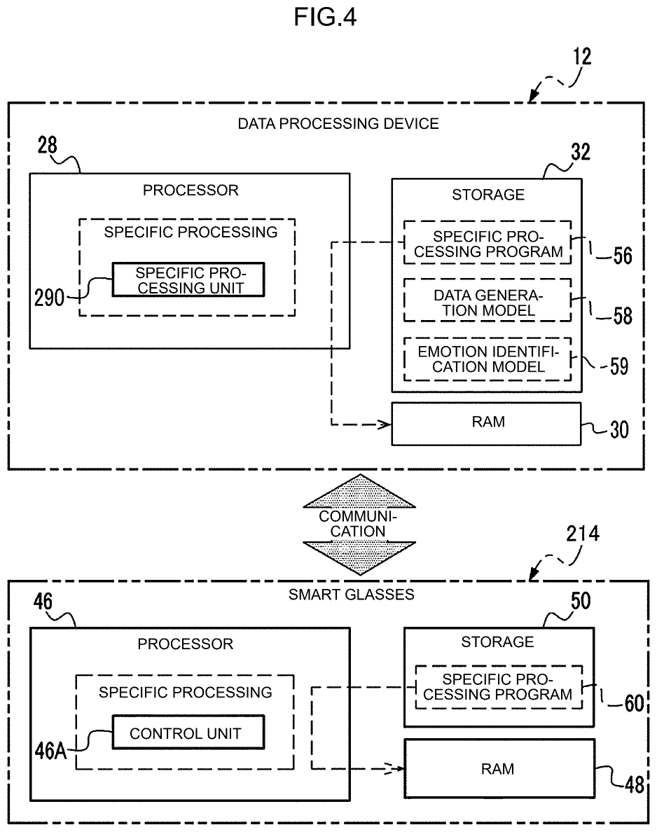

FIG. 4 shows an example of the main functions of the data processing device 12 and smart glasses 214. As shown in FIG. 4, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart glasses 214, specific processing is performed by the processor 46. The storage 50 stores a specific processing program 60. The processor 46 reads the specific processing program 60 from the storage 50 and executes it on the RAM 48. The specific processing is realized by the processor 46 operating as a control unit 46A according to the specific processing program 60 executed on the RAM 48. The smart glasses 214 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the smart glasses 214. In the smart glasses 214, the control unit 46A causes the speaker 240 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 210 according to the second embodiment performs the same processing as the data processing system 10 according to the first embodiment. The processing by the data processing system 210 is executed by the specific processing unit 290 of the data processing device 12 or the control unit 46A of the smart glasses 214, but it may be executed by both the specific processing unit 290 of the data processing device 12 and the control unit 46A of the smart glasses 214. Additionally, the specific processing unit 290 of the data processing device 12 acquires or collects necessary information for processing from the smart glasses 214 or external devices, and the smart glasses 214 acquires or collects necessary information for processing from the data processing device 12 or external devices.

Each of the plurality of elements including the aforementioned recording unit, conversion unit, analysis unit, and provision unit is implemented by at least one of, for example, the smart glasses 214 and the data processing apparatus 12. For example, the recording unit records the user's voice using the microphone 238 of the smart glasses 214. The conversion unit converts the voice into a character string by, for example, a specific processing unit 290 of the data processing apparatus 12. The analysis unit analyzes the character string by, for example, the specific processing unit 290 of the data processing apparatus 12 and identifies statements that impair psychological safety. The provision unit provides the analysis result as a report by, for example, a control unit 46A of the smart glasses 214. The correspondence between each unit and the device or control unit is not limited to the examples described above and various modifications are possible.

### Third Embodiment

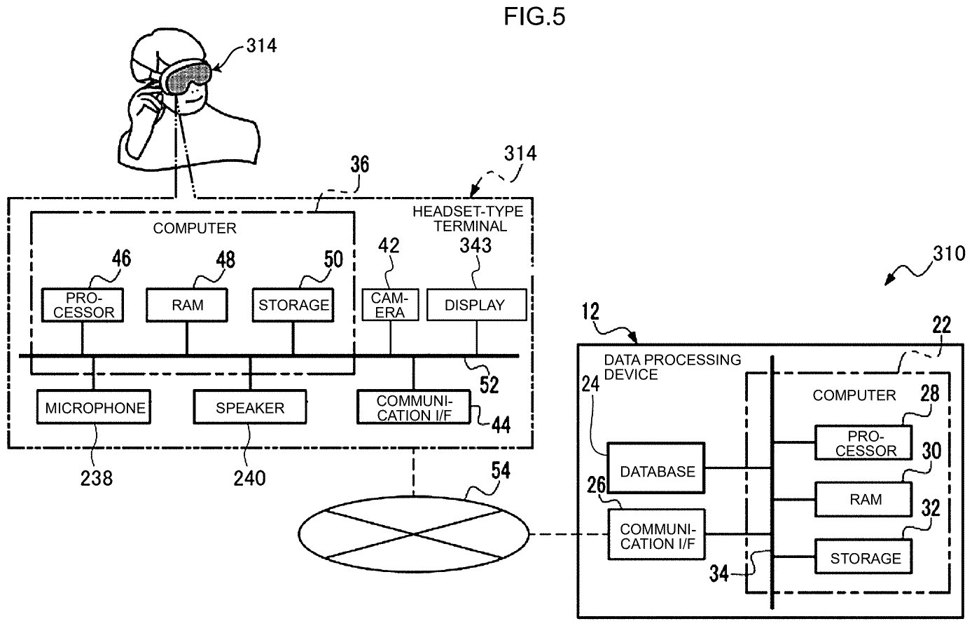

FIG. 5 shows an example configuration of a data processing system 310 according to the third embodiment.

As shown in FIG. 5, the data processing system 310 comprises a data processing device 12 and a headset-type terminal 314. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The headset-type terminal 314 comprises a computer 36, a microphone 238, a speaker 240, a camera 42, a communication I/F 44, and a display 343. The computer 36 comprises a processor 46, RAM 48, and storage 50. The processor 46, RAM 48, and storage 50 are connected to a bus 52. The microphone 238, speaker 240, camera 42, and display 343 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

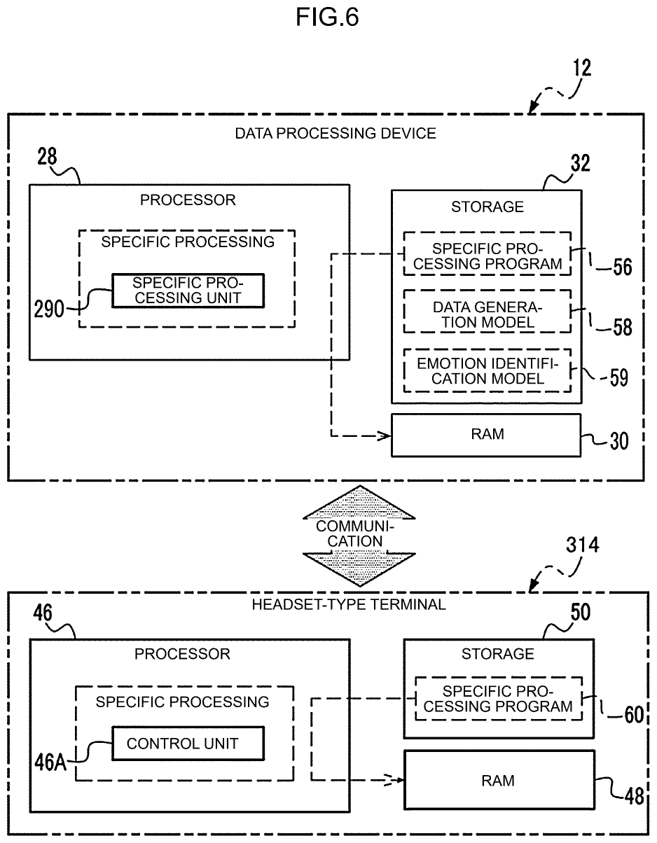

FIG. 6 shows an example of the main functions of the data processing device 12 and the headset-type terminal 314. As shown in FIG. 6, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the headset-type terminal 314, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The headset-type terminal 314 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the headset-type terminal 314. In the headset-type terminal 314, the control unit 46A causes the speaker 240 and the display 343 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 310 according to the third embodiment performs the same processing as the data processing system 10 according to the first embodiment. The processing by the data processing system 310 is executed by the specific processing unit 290 of the data processing device 12 or the control unit 46A of the headset-type terminal 314, but it may be executed by both the specific processing unit 290 of the data processing device 12 and the control unit 46A of the headset-type terminal 314. Additionally, the specific processing unit 290 of the data processing device 12 acquires or collects necessary information for processing from the headset-type terminal 314 or external devices, and the headset-type terminal 314 acquires or collects necessary information for processing from the data processing device 12 or external devices.

Each of the plurality of elements including the aforementioned recording unit, conversion unit, analysis unit, and provision unit is implemented by at least one of, for example, the headset-type terminal 314 and the data processing apparatus 12. For example, the recording unit records the user's voice using the microphone 238 of the headset-type terminal 314. The conversion unit converts the voice into a character string by, for example, a specific processing unit 290 of the data processing apparatus 12. The analysis unit analyzes the character string by, for example, the specific processing unit 290 of the data processing apparatus 12 and identifies statements that impair psychological safety. The provision unit provides the analysis result as a report by, for example, a control unit 46A of the headset-type terminal 314. The correspondence between each unit and the device or control unit is not limited to the examples described above and various modifications are possible.

### Fourth Embodiment

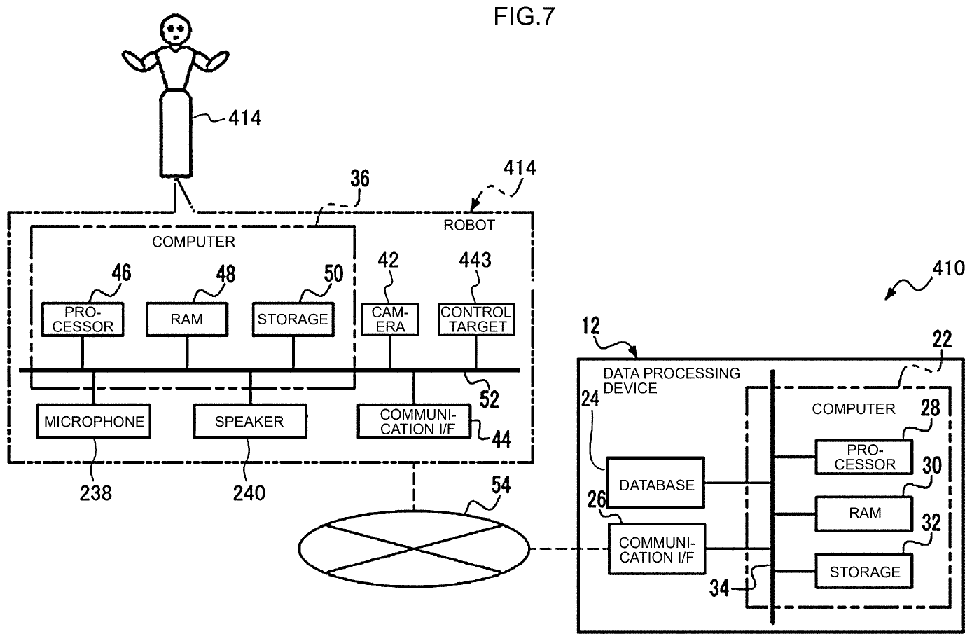

FIG. 7 shows an example configuration of a data processing system 410 according to the fourth embodiment.

As shown in FIG. 7, the data processing system 410 comprises a data processing device 12 and a robot 414. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The robot 414 comprises a computer 36, a microphone 238, a speaker 240, a camera 42, a communication I/F 44, and a control target 443. The computer 36 comprises a processor 46, RAM 48, and storage 50. The processor 46, RAM 48, and storage 50 are connected to a bus 52. The microphone 238, speaker 240, camera 42, and control target 443 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS image sensors or CCD image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

The control target 443 includes a display device, LEDs for the eyes, and motors for driving arms, hands, and feet, among others. The posture and gestures of the robot 414 are controlled by controlling the motors for the arms, hands, and feet, among others. Some emotions of the robot 414 can be expressed by controlling these motors. Additionally, the expression of the robot 414 can be expressed by controlling the lighting state of the LEDs for the eyes of the robot 414.

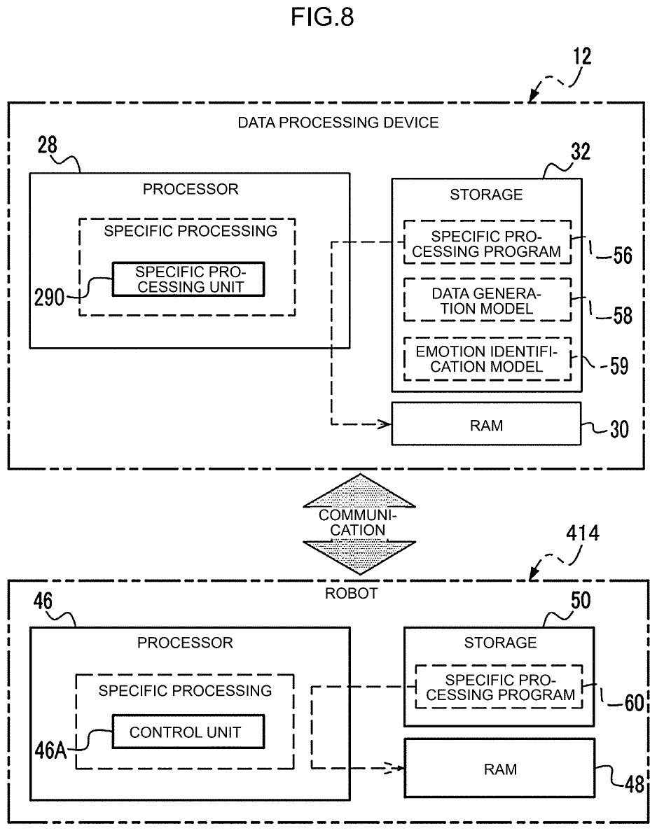

FIG. 8 shows an example of the main functions of the data processing device 12 and the robot 414. As shown in FIG. 8, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the robot 414, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The robot 414 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the robot 414. In the robot 414, the control unit 46A causes the speaker 240 and the control target 443 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 410 according to the fourth embodiment performs the same processing as the data processing system 10 according to the first embodiment. The processing by the data processing system 410 is executed by the specific processing unit 290 of the data processing device 12 or the control unit 46A of the robot 414, but it may be executed by both the specific processing unit 290 of the data processing device 12 and the control unit 46A of the robot 414. Additionally, the specific processing unit 290 of the data processing device 12 acquires or collects necessary information for processing from the robot 414 or external devices, and the robot 414 acquires or collects necessary information for processing from the data processing device 12 or external devices.

Each of the plurality of elements including the aforementioned recording unit, conversion unit, analysis unit, and provision unit is implemented by at least one of, for example, the robot 414 and the data processing apparatus 12. For example, the recording unit records the user's voice using the microphone 238 of the robot 414. The conversion unit converts the voice into a character string by, for example, a specific processing unit 290 of the data processing apparatus 12. The analysis unit analyzes the character string by, for example, the specific processing unit 290 of the data processing apparatus 12 and identifies statements that impair psychological safety. The provision unit provides the analysis result as a report by, for example, a control unit 46A of the robot 414. The correspondence between each unit and the device or control unit is not limited to the examples described above and various modifications are possible.

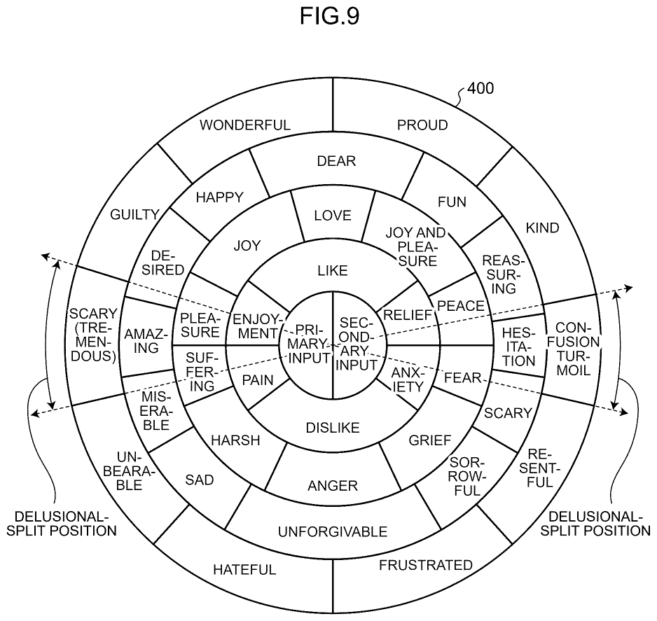

Note that the emotion identification model 59 as an emotion engine may determine the user's emotions according to a specific mapping. Specifically, the emotion identification model 59 may determine the user's emotions according to an emotion map, which is a specific mapping (see FIG. 9). Similarly, the emotion identification model 59 may determine the robot's emotions, and the specific processing unit 290 may perform specific processing using the robot's emotions.

FIG. 9 is a diagram showing an emotion map 400 where multiple emotions are mapped. In the emotion map 400, emotions are arranged concentrically radiating from the center. The closer to the center of the concentric circles, the more primitive the state of emotions is arranged. On the outer side of the concentric circles, emotions representing states and behaviors arising from mood are arranged. Emotions encompass concepts including emotional and mental states. On the left side of the concentric circles, emotions generally generated from reactions occurring in the brain are arranged. On the right side of the concentric circles, emotions generally induced by situational judgment are arranged. On the top and bottom of the concentric circles, emotions generated from reactions occurring in the brain and induced by situational judgment are arranged. Additionally, on the upper side of the concentric circles, “pleasant” emotions are arranged, and on the lower side, “unpleasant” emotions are arranged. In this way, in the emotion map 400, multiple emotions are mapped based on the structure from which emotions arise, and emotions that tend to occur simultaneously are mapped nearby.

These emotions are distributed in the 3 o'clock direction of the emotion map 400, and they usually move back and forth around reassurance and anxiety. In the right half of the emotion map 400, situational recognition takes precedence over internal sensations, giving a calm impression.

The inner side of the emotion map 400 represents the mind, and the outer side represents behavior, so the further out on the emotion map 400, the more visible (expressed in behavior) emotions become.

Here, human emotions are based on various balances like posture and blood sugar levels, and when these balances move away from the ideal, they indicate discomfort, and when they approach the ideal, they indicate comfort. In robots, cars, motorcycles, etc., emotions can be created based on various balances like posture and battery level, indicating discomfort when these balances move away from the ideal and comfort when they approach the ideal. The emotion map may be generated based on Dr. Mitsuyoshi's emotion map (Research on speech emotion recognition and brain physiological signal analysis systems related to emotions, Tokushima University, Doctoral dissertation: https://ci.nii.ac.jp/naid/500000375379). In the left half of the emotion map, emotions belonging to the domain called “reactions,” where sensations take precedence, are aligned. Additionally, in the right half of the emotion map, emotions belonging to the domain called “situations,” where situational recognition takes precedence, are aligned.

In the emotion map, two emotions that promote learning are defined. One is a negative emotion around “repentance” or “reflection” on the situation side. In other words, when a negative emotion arises in the robot, like “I never want to feel this way again” or “I don't want to be scolded again.” The other is an emotion around “desire” on the reaction side, which is positive. In other words, it is a positive feeling like “I want more” or “I want to know more.”

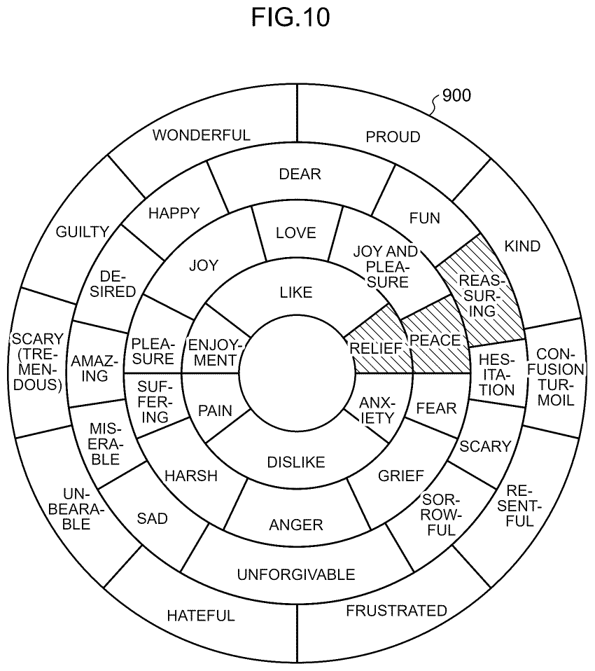

The emotion identification model 59 inputs user input into a pre-learned neural network, acquires emotion values indicating each emotion shown in the emotion map 400, and determines the user's emotions. This neural network is pre-learned based on multiple training data consisting of user input and combinations of emotion values indicating each emotion shown in the emotion map 400. Additionally, this neural network is learned so that emotions placed near each other in the emotion map 900 shown in FIG. 10 have similar values. FIG. 10 shows an example where multiple emotions like “reassured,” “calm,” and “confident” have similar emotion values.

In the above embodiments, an example form where specific processing is performed by a single computer 22 was described, but the technology disclosed herein is not limited to this, and distributed processing for specific processing by multiple computers including the computer 22 may be performed.

In the above embodiments, an example form where the specific processing program 56 is stored in the storage 32 was described, but the technology disclosed herein is not limited to this. For example, the specific processing program 56 may be stored in portable non-transitory storage media readable by a computer, such as a USB (Universal Serial Bus) memory. The specific processing program 56 stored in non-transitory storage media is installed in the computer 22 of the data processing device 12. The processor 28 executes specific processing according to the specific processing program 56.

Additionally, the specific processing program 56 may be stored in a storage device, such as a server connected to the data processing device 12 via the network 54, and downloaded and installed on the computer 22 in response to requests from the data processing device 12.

Furthermore, it is not necessary to store all of the specific processing program 56 in storage devices such as servers connected to the data processing device 12 via the network 54 or all in the storage 32, and a part of the specific processing program 56 may be stored.

Various processors, as shown next, can be used as hardware resources for executing specific processing. As processors, general-purpose processors that function as hardware resources for executing specific processing by executing software, i.e., programs, such as a CPU, can be mentioned. Additionally, as processors, dedicated electrical circuits with circuit configurations specially designed to execute specific processing, such as FPGA (Field-Programmable Gate Array), PLD (Programmable Logic Device), or ASIC (Application Specific Integrated Circuit), can be mentioned. Each processor has a built-in or connected memory, and each processor executes specific processing using the memory.

Hardware resources for executing specific processing may be composed of one of these various processors or a combination of two or more processors of the same or different types (e.g., a combination of multiple FPGAs or a combination of a CPU and FPGA). Additionally, hardware resources for executing specific processing may be a single processor.

As an example of composing with a single processor, firstly, there is a form where one or more CPUs and software are combined to constitute a single processor, which functions as hardware resources for executing specific processing. Secondly, there is a form using a processor, such as SoC (System-on-a-chip), that realizes the function of an entire system including multiple hardware resources for executing specific processing with a single IC chip. In this way, specific processing is realized using one or more of the various processors as hardware resources.

Furthermore, as a hardware structure of these various processors, more specifically, electrical circuits combined with circuit elements such as semiconductor elements can be used. Additionally, the specific processing described above is merely one example. Therefore, it goes without saying that unnecessary steps may be deleted, new steps may be added, or the order of processing may be changed within the scope not departing from the gist.

Additionally, in the examples described above, the explanation was divided into the first embodiment to the fourth embodiment, but parts or all of these embodiments may be combined. Additionally, the smart device 14, smart glasses 214, headset-type terminal 314, and robot 414 are examples, and each may be combined, or other devices may be used.

The descriptions and drawings shown above are detailed explanations of parts related to the technology disclosed herein and are merely examples of the technology disclosed herein. For example, the explanations regarding configurations, functions, actions, and effects above are explanations regarding examples of configurations, functions, actions, and effects of parts related to the technology disclosed herein. Therefore, it goes without saying that within the scope not departing from the gist of the technology disclosed herein, unnecessary parts may be deleted, new elements may be added, or replacements may be made to the descriptions and drawings shown above. Additionally, to avoid complexity and facilitate understanding of parts related to the technology disclosed herein, explanations concerning technical common knowledge and the like that do not require special explanation for enabling the implementation of the technology disclosed herein are omitted in the descriptions and drawings shown above.

All documents, patent applications, and technical standards described in this specification are incorporated by referenwce to the same extent as if each document, patent application, and technical standard were specifically and individually stated to be incorporated by reference in this specification.
(Supplementary Note 1) A system comprising: a recording unit configured to record a user's voice; a conversion unit configured to convert the voice recorded by the recording unit into a character string; an analysis unit configured to analyze the character string converted by the conversion unit; and a provision unit configured to provide the analysis result obtained by the analysis unit as a report.
(Supplementary Note 2) The system according to Supplementary Note 1, wherein the recording unit is configured to record the user's voiceprint.
(Supplementary Note 3) The system according to Supplementary Note 1, wherein the conversion unit is configured to convert the recorded voice into a character string.
(Supplementary Note 4) The system according to Supplementary Note 1, wherein the analysis unit describes a specific method for identifying statements that impair psychological safety.
(Supplementary Note 5) The system according to Supplementary Note 1, wherein the provision unit is configured to provide the analysis result as a report.
(Supplementary Note 6) The system according to Supplementary Note 1, wherein the provision unit is configured to provide information for the user to learn by reading the report.
(Supplementary Note 7) The system according to Supplementary Note 1, wherein the provision unit describes a specific method for saving more than ten records in a paid version.
(Supplementary Note 8) The system according to Supplementary Note 1, wherein the provision unit is configured to confirm the growth trend of communication skills related to psychological safety.
(Supplementary Note 9) The system according to Supplementary Note 1, wherein the recording unit is configured to estimate the user's emotion and adjust the timing of voice recording based on the estimated emotion of the user.
(Supplementary Note 10) The system according to Supplementary Note 1, wherein the recording unit is configured to analyze the user's past conversation history and select an optimal recording method.
(Supplementary Note 11) The system according to Supplementary Note 1, wherein the recording unit is configured to filter the user's current environmental sounds and remove noise during voice recording.
(Supplementary Note 12) The system according to Supplementary Note 1, wherein the recording unit is configured to estimate the user's emotion and determine the priority of the voice to be recorded based on the estimated emotion of the user.
(Supplementary Note 13) The system according to Supplementary Note 1, wherein the recording unit describes a specific method for preferentially recording highly relevant voice by considering the user's geographic location information during voice recording.
(Supplementary Note 14) The system according to Supplementary Note 1, wherein the recording unit describes a specific method for analyzing the user's social media activity and recording relevant voice during voice recording.
(Supplementary Note 15) The system according to Supplementary Note 1, wherein the conversion unit is configured to estimate the user's emotion and adjust the conversion accuracy of the character string based on the estimated emotion of the user.
(Supplementary Note 16) The system according to Supplementary Note 1, wherein the conversion unit is configured to improve conversion accuracy by considering the context of the conversation during voice conversion.
(Supplementary Note 17) The system according to Supplementary Note 1, wherein the conversion unit is configured to apply conversion algorithms corresponding to different languages and dialects during voice conversion.
(Supplementary Note 18) The system according to Supplementary Note 1, wherein the conversion unit is configured to estimate the user's emotion and adjust the length of the character string to be converted based on the estimated emotion of the user.
(Supplementary Note 19) The system according to Supplementary Note 1, wherein the conversion unit is configured to determine the priority of conversion based on the importance of the conversation during voice conversion.
(Supplementary Note 20) The system according to Supplementary Note 1, wherein the conversion unit is configured to adjust the order of conversion based on the relevance of the conversation during voice conversion.
(Supplementary Note 21) The system according to Supplementary Note 1, wherein the analysis unit is configured to estimate the user's emotion and adjust the criteria for analysis based on the estimated emotion of the user.
(Supplementary Note 22) The system according to Supplementary Note 1, wherein the analysis unit describes a specific method for improving the accuracy of analysis by considering the interrelationship of conversations during analysis.
(Supplementary Note 23) The system according to Supplementary Note 1, wherein the analysis unit describes a specific method for analyzing by considering attribute information of conversation participants during analysis.
(Supplementary Note 24) The system according to Supplementary Note 1, wherein the analysis unit is configured to estimate the user's emotion and adjust the display order of the analysis result based on the estimated emotion of the user.
(Supplementary Note 25) The system according to Supplementary Note 1, wherein the analysis unit describes a specific method for analyzing by considering the geographic distribution of conversations during analysis.
(Supplementary Note 26) The system according to Supplementary Note 1, wherein the analysis unit describes a specific method for improving the accuracy of analysis by referring to related literature of the conversation during analysis.
(Supplementary Note 27) The system according to Supplementary Note 1, wherein the provision unit is configured to estimate the user's emotion and adjust the display method of the report based on the estimated emotion of the user.
(Supplementary Note 28) The system according to Supplementary Note 1, wherein the provision unit is configured to optimize the current report by referring to past report data when providing the report.
(Supplementary Note 29) The system according to Supplementary Note 1, wherein the provision unit is configured to apply different report formats for each category of conversation when providing the report.
(Supplementary Note 30) The system according to Supplementary Note 1, wherein the provision unit is configured to estimate the user's emotion and adjust the importance of the report based on the estimated emotion of the user.
(Supplementary Note 31) The system according to Supplementary Note 1, wherein the provision unit is configured to determine the priority of the report based on the submission timing of the conversation when providing the report.
(Supplementary Note 32) The system according to Supplementary Note 1, wherein the provision unit is configured to analyze the report by referring to related market data of the conversation when providing the report.
(Supplementary Note 33) The system according to Supplementary Note 1, wherein the provision unit (paid version) is configured to estimate the user's emotion and determine the priority of records to be saved based on the estimated emotion of the user.
(Supplementary Note 34) The system according to Supplementary Note 1, wherein the provision unit (paid version) is configured to optimize the saving algorithm by referring to past saved data when saving records.
(Supplementary Note 35) The system according to Supplementary Note 1, wherein the provision unit (paid version) is configured to estimate the user's emotion and adjust the display method of records to be saved based on the estimated emotion of the user.
(Supplementary Note 36) The system according to Supplementary Note 1, wherein the provision unit (paid version) is configured to weight the saved data based on the submission timing of the conversation when saving records.
(Supplementary Note 37) The system according to Supplementary Note 1, wherein the provision unit (growth trend confirmation) is configured to estimate the user's emotion and adjust the display method of the growth trend based on the estimated emotion of the user.
(Supplementary Note 38) The system according to Supplementary Note 1, wherein the provision unit (growth trend confirmation) is configured to optimize the current growth by referring to past growth data when confirming the growth trend.
(Supplementary Note 39) The system according to Supplementary Note 1, wherein the provision unit (growth trend confirmation) is configured to estimate the user's emotion and determine the priority of the growth trend based on the estimated emotion of the user.
(Supplementary Note 40) The system according to Supplementary Note 1, wherein the provision unit (growth trend confirmation) is configured to weight the growth data based on the submission timing of the conversation when confirming the growth trend.

What is claimed is:

1. A system comprising:
a communication interface configured to communicate with a client terminal via a packet-switched network;
a memory storing a speech recognition model and a natural language processing model, each obtained by machine learning;
a database; and
circuitry configured to:
receive, from the client terminal via the communication interface, audio data representing a voice of a user captured by a microphone of the client terminal;
convert the received audio data into a character string by inputting the audio data into the speech recognition model;
analyze the character string by inputting the character string into the natural language processing model to identify, for each statement in the character string, a psychological safety risk score indicating a degree to which the statement impairs psychological safety; and
generate a report based on the psychological safety risk score for each statement, and transmit the report to the client terminal via the communication interface and the packet-switched network.

2. The system according to claim 1, wherein the circuitry is further configured to extract a voiceprint feature vector from the audio data by inputting the audio data into a voiceprint extraction model, and store the voiceprint feature vector in the database as a user identifier.

3. The system according to claim 1, wherein the speech recognition model comprises at least one of a convolutional neural network, a recurrent neural network, or a Transformer-based model, and wherein converting the received audio data comprises extracting acoustic features from the audio data and determining an optimal character string sequence using at least one of a beam search decoder or a connectionist temporal classification decoder.

4. The system according to claim 1, wherein the natural language processing model comprises a Transformer-based large-scale language model, and wherein analyzing the character string comprises tokenizing the character string into a token sequence, converting the token sequence into embedding vectors, and classifying each statement as at least one of safe, aggressive, or negative.

5. The system according to claim 1, wherein the report comprises at least one of a risk assessment for each statement, an improvement example for each statement identified as impairing psychological safety, or a comparison graph showing a time-series transition of psychological safety scores.

6. The system according to claim 1, wherein the report is generated in at least one of a PDF format, an HTML format, or a JSON format.

7. The system according to claim 1, wherein the circuitry is further configured to estimate an emotion of the user based on the audio data, and adjust a parameter of the speech recognition model based on the estimated emotion, the parameter comprising at least one of a beam search width or a noise suppression level.

8. The system according to claim 1, wherein the circuitry is further configured to convert the received audio data into the character string by further inputting, into the speech recognition model, text data of a conversation history comprising a character string sequence of previous sentences, and wherein the speech recognition model processes acoustic features and contextual features using a multi-layer attention mechanism.

9. The system according to claim 1, wherein the circuitry is further configured to identify a language or a dialect of the audio data, and select a speech recognition model corresponding to the identified language or dialect from a plurality of speech recognition models stored in the memory.

10. The system according to claim 1, wherein the circuitry is further configured to estimate an emotion of the user based on the audio data, and adjust a length of the character string based on the estimated emotion by controlling at least one of a summarization rate or a detail rate of the speech recognition model.

11. The system according to claim 1, wherein the circuitry is further configured to compute an importance score for the audio data using a natural language processing model, and determine a conversion priority for the audio data based on the importance score, the conversion priority being one of priority, normal, or deferred.

12. The system according to claim 1, wherein the circuitry is further configured to estimate an emotion of the user based on the audio data, and adjust a threshold for the psychological safety risk score based on the estimated emotion, such that the threshold is stricter when the estimated emotion indicates nervousness and relaxed when the estimated emotion indicates urgency.

13. The system according to claim 1, wherein the circuitry is further configured to analyze dependencies between statements in the character string using a multi-layer attention mechanism or a graph neural network to determine, for each statement, an intent label and an impact score.

14. The system according to claim 1, wherein the circuitry is further configured to receive attribute information of conversation participants, the attribute information comprising at least one of age, occupation, or position, and adjust an impact score for each statement based on the attribute information.

15. The system according to claim 1, wherein the circuitry is further configured to receive geographic location information associated with the audio data, and analyze a statement trend for each geographic region based on the geographic location information and the character string.

16. The system according to claim 1, wherein the circuitry is further configured to estimate an emotion of the user and adjust a display method of the report based on the estimated emotion, the display method comprising at least one of a simple layout when the estimated emotion indicates nervousness, a detailed layout with graphs and annotations when the estimated emotion indicates relaxation, or a summary layout emphasizing key points when the estimated emotion indicates urgency.

17. The system according to claim 1, wherein the circuitry is further configured to retrieve past report data for the user from the database, and optimize a format of the report based on the past report data by analyzing report content trends and user preferences extracted from the past report data.

18. A system comprising:
a communication interface configured to communicate with a client terminal via a packet-switched network, the communication interface supporting at least one of a 5G, Wi-Fi, or Bluetooth communication standard;
a memory storing a speech recognition model comprising a Transformer-based encoder-decoder architecture obtained by deep learning on a neural network, and a natural language processing model comprising a Transformer-based large-scale language model;
a database; and
circuitry comprising at least one of a CPU, a GPU, or a TPU, the circuitry configured to:
receive, from the client terminal via the communication interface, audio data representing a voice of a user captured by a microphone of the client terminal;
extract acoustic features from the audio data, the acoustic features comprising at least one of mel-frequency cepstral coefficients or a spectrogram;
convert the acoustic features into a character string by determining an optimal character string sequence using at least one of a beam search decoder or a connectionist temporal classification decoder of the speech recognition model;
tokenize the character string into a token sequence and analyze the token sequence using the natural language processing model to identify, for each statement in the character string, a psychological safety risk score and a label indicating whether the statement is safe, aggressive, or negative;
generate a report in at least one of a PDF format, an HTML format, or a JSON format, the report comprising a risk assessment for each statement and an improvement example for each statement identified as impairing psychological safety; and
transmit the report to the client terminal via the communication interface and the packet-switched network, the report causing the client terminal to present the report to the user.

19. The system according to claim 18, wherein the memory further stores an emotion identification model, and wherein the circuitry is further configured to estimate an emotion of the user by inputting the audio data into the emotion identification model, and adjust at least one of: a beam search width of the speech recognition model, a threshold for the psychological safety risk score, or a display method of the report, based on the estimated emotion.

20. A method performed by a system comprising a communication interface, a memory, a database, and circuitry, the method comprising:
receiving, from a client terminal via the communication interface and a packet-switched network, audio data representing a voice of a user captured by a microphone of the client terminal;
converting the received audio data into a character string by inputting the audio data into a speech recognition model stored in the memory;
analyzing the character string by inputting the character string into a natural language processing model stored in the memory to identify, for each statement in the character string, a psychological safety risk score indicating a degree to which the statement impairs psychological safety;
generating a report based on the psychological safety risk score for each statement; and
transmitting the report to the client terminal via the communication interface and the packet-switched network.
